(19) United States
(12) Patent Application Publication
(10) Pub. No.: US 2014/0356287 A1
(43) Pub. Date: Dec. 4, 2014

(54) PLOD2 as a Target of Intervention for Sarcoma Metastasis

(71) Applicant: The Trustees of the University of Pennsylvania, Philadelphia, PA (US)

(72) Inventors: M. Celeste Simon, Philadelphia, PA (US); T.S. Karin Eisinger, Philadelphia, PA (US)

(73) Assignee: The Trustees of the University of Pennsylvania, Philadelphia, PA (US)

(21) Appl. No.: 14/287,752

(22) Filed: May 27, 2014

Related U.S. Application Data
(60) Provisional application No. 61/828,028, filed on May 28, 2013.

(51) Int. Cl. A61K 49/00; C12N 15/11; A61K 38/17; A61K 31/506; C12N 15/113; C07K 16/18

(52) U.S. Cl. CPC A61K 49/0004; C12N 15/113; C07K 16/18; A61K 38/17; A61K 31/506; C12N 15/11; A61K 2039/505
USPC 424/9.1; 514/44 A; 424/174.1; 514/19.3; 514/275; 514/44 R

(57) ABSTRACT

The invention provides compositions and methods for treating a disease or disorder by lowering the level of PLOD2 in a subject.

### CROSS-REFERENCE TO RELATED APPLICATIONS

This application claims priority to U.S. Provisional Application Ser. No. 61/828,028, filed May 28, 2013, the content of which is incorporated by reference herein in its entirety.

### STATEMENT REGARDING FEDERALLY SPONSORED RESEARCH OR DEVELOPMENT

This invention was made with government support under CA156979 and CA158301 awarded by the National Cancer Institute (NCI). The government has certain rights in the invention.

### BACKGROUND OF THE INVENTION

Sarcomas are diagnosed in 200,000 people worldwide annually; approximately 40% of whom ultimately succumb to lethal metastases (Jemal et al., 2010, CA Cancer J Clin 60(5):277-300; Italiano et al., 2011, Cancer 117(5):1049-54). Current treatment options available to sarcoma patients are standard surgical resection, radiation, and chemotherapy, and limited molecular analyses of human sarcomas have proven an impediment to developing novel, sarcoma-specific, therapeutic options. Although genetic lesions affecting multiple signaling pathways (Kras, Pten, Ptch1, p. 53) have been identified in distinct soft tissue sarcoma subtypes (Soleimani et al., 2011, Cancer Cell 19(2):157-9; Rubin et al., 2011, Cancer Cell 19(2):177-91), relatively little is understood about the downstream molecular mechanisms that drive sarcomagenesis and progression. Furthermore, soft tissue sarcomas encompass more than 50 distinct disease subtypes (e.g. fibrosarcoma, liposarcoma, rhabdomyosarcoma, etc.), all of which undergo constant reevaluation as developing technologies allow for more thorough characterization of each malignancy (Borden et al., 2003, Clin Cancer Res 9(6):1941-56; Kattan et al., 2002, J Clin Oncol 20(3):791-6). As in other tumors, aggressive metastatic behavior in sarcomas is frequently associated with high levels of tumor cell dedifferentiation (Helman and Meltzer, 2003, Nat Rev Cancer 3(9)685-94). Consistent with this observation, Undifferentiated Pleomorphic Sarcoma (UPS) has been identified as one of the most frequently diagnosed subtypes, and commonly results in lethal pulmonary metastases.

Current data suggest that UPS may not represent a distinct sarcoma subtype, but rather a collection of phenotypes common to other sarcomas in their more advanced stages (Matushansky et al., 2009, Expert Rev Anticancer Ther 9(8):1135-44). It has been argued that UPS exists along a continuum wherein unique sarcoma subtypes become increasingly undifferentiated as they worsen in stage and grade until their tissue/cell type of origin is no longer discernible (Matushansky et al., 2009, Expert Rev Anticancer Ther 9(8):1135-44). Regardless of whether UPS is ultimately shown to be distinct or a culmination of sarcoma progression, these tumors are associated with poor clinical outcome due to metastases. As metastasis, particularly to the lungs, remains the most common cause of sarcoma-associated death, elucidating the molecular and cellular mechanisms controlling sarcoma cell dissemination is critical to the development of effective therapeutic strategies to treat these cancers.

The available clinical data indicate that high levels of intratumoral hypoxia and HIF1α expression are among the most important predictors of metastatic potential in sarcoma patients, although the underlying mechanisms for this correlation are unknown (Brizel et al., 1996, Cancer Res 56(5):941-3; Maseide et al., 2004, Clin Cancer Res 10(13):4464-71; Nordsmark et al., 2001, Br J Cancer 84(8):1070-5). Metastasis is a complex multistep process wherein tumor cells are driven, in part by lack of oxygen and nutrients, to abandon their tissue of origin and colonize distant sites (Cairns et al., 2003, Curr Mol Med 3(7):659-71; Wong et al., 2012, J Mol Med (Berl) 90(7):803-15; Erler et al., 2006, Nature 440(7088):1222-6). For example, hypoxia has been shown to promote release of tumor cell-derived lysyl oxidase (LOX), a HIF1α target that remodels collagen in the extracellular matrix of remote sites, thereby contributing to the establishment of the “pre-metastatic niche” (Peyrol et al., 1997, Am J Pathol 150(2):497-507; Santhanam et al., 2010, Oncogene 29(27):3921-32) in murine breast cancer models. Whether similar, or distinct, cellular mechanisms regulate sarcoma metastasis is as yet unknown.

Collagen is the most abundant structural component of the extracellular matrix (ECM) and is aberrantly regulated in cancer at the levels of expression, post-translational modification, deposition and degradation (Jodele et al., 2006, Cancer Metastasis Rev 25(1):35-43). Consistent with their mesenchymal origins, primary sarcomas produce and secrete large amounts of collagen, generating extensive extracellular collagen “highways” (Pihlajaniemi et al., 1981, Biochemistry 20(26):7409-15). These networks act as support scaffolds, facilitating tumor cell migration toward blood vessels and promoting their ability to escape the primary lesion (Egeblad et al., 2010, Curr Opin Cell Biol 22(5):597-706; Wang et al., 2002, Cancer Res 62(21):6278-88; Wyckoff et al., 2007, Cancer Res 67(6):2649-56; Zaman et al., 2006, Proc Natl Acad Sci USA 103(29):10889-94; Condeelis et al., 2003, Nat Rev Cancer 3(12):921-30; Han et al., 2010, J Biol Chem 285(29):22276-81; Levental et al., 2009, Cell 139(5):891-906; Makareeva et al., 2010, Cancer Res 70(11):4366-74). Mature collagen is formed by a series of enzymatic post-translational modifications of immature collagen polypeptides (Pihlajaniemi et al., 1981, Biochemistry 20(26):7409-15; Myllyharju and Kivirikko, 2004, Trends Genet 20(1):33-43; Sipila et al., 2007, J Biol Chem 282(46):33381-8), although the factors required to establish and maintain collagen networks in sarcomas are not clear. Recently, HIF1α has been shown to regulate expression of the endoplasmic reticulum (ER)-associated enzyme procollagen-lysine, 2-oxoglutarate 5-dioxygenase (PLOD2), also referred to as lysyl hydroxylase 2 (LH2) (Erler et al., 2006, Nature 440(7088):1222-6; Hofbauer et al., 2003, Eur J Biochem 270(22):4515-22; Erler and Giaccia, 2006, Cancer Res 66(21):10238-41). The primary function of PLOD2 is the initiation of lysine hydroxylation of collagen molecules (Rautavuoma et al., 2002, J Biol Chem 277(25):23084-91; Pirskanen et al., 1996, J Biol Chem 271(16):9398-402; Hyry et al., 2009, J Biol Chem 284(45):30917-24). Hydroxylysines form carbohydrate attachment sites and are essential for the stability of collagen crosslinks. Crosslinked collagen assembles into a triple helix, departs the ER and is then cleaved for assembly into fibrils (Myllyharju and Kivirikko, 2004, Trends Genet 20(1):33-43). Prolyl and lysyl hydroxylation are crucial for the formation of normal mature collagen. Mutations in PLOD2 cause the autosomal recessive disorder, Bruck syndrome, in which patients suffer osteoporosis, scoliosis, and joint contractures due to underhydroxylated collagen I (Hyry et al., 2009, J Biol Chem 284(45):30917-24); however, very little is known about the role of PLOD2 in tumorigenesis. Furthermore, the majority of research investigating the contribution of collagen and collagen-modifying enzymes to metastasis has been performed on epithelial cell-derived tumors, primarily breast cancer (Santhanam et al., 2010, Oncogene 29(27):3921-32; Erler and Giaccia, 2006, Cancer Res 66(21):10238-41; Gilkes et al., 2013 J Biol Chem 288(15):10819-29). These processes remain understudied in mesenchymal tumors, including sarcomas.

Undifferentiated pleomorphic sarcomas (UPS) is a commonly diagnosed and aggressive sarcoma subtype in adults, which frequently and fatally metastasizes to the lung. Thus, there is an urgent need in the art for compositions and methods for diagnosing and treating sarcomas. The present invention addresses this need.

### SUMMARY OF THE INVENTION

The invention provides a method for interfering with at least one of HIF1α and a collagen modifying enzyme. In one embodiment, the method comprises administering to a subject in need thereof an effective amount of a composition comprising an inhibitor of at least one of HIF1α and a collagen modifying enzyme.

In one embodiment, the collagen modifying enzyme is selected from the group consisting of procollagen-lysine 5-dioxygenase 1 (PLOD1); procollagen-lysine 2-oxoglutarate 5-dioxygenase 2 (PLOD2); procollagen-lysine 5-dioxygenase 3 (PLOD3) and any combination thereof.

In one embodiment, the interfering with a collagen modifying enzyme comprises one or more of the level of the collagen modifying enzyme and the activity of the collagen modifying enzyme.

In one embodiment, the inhibitor prevents the transcription of the collagen modifying enzyme gene or translation of the collagen modifying enzyme mRNA.

In one embodiment, the inhibitor interferes with the activity of the collagen modifying enzyme.

In one embodiment, the inhibitor is selected from the group consisting of a small interfering RNA (siRNA), a microRNA, an antisense nucleic acid, a ribozyme, an expression vector encoding a transdominant negative mutant, an antibody, a peptide and a small molecule.

In one embodiment, the inhibitor is minoxidil or a salt or chemical analog thereof.

In one embodiment, the collagen modifying enzyme is associated with at least one of cancer metastasis, cancer cell growth, cancer invasion, and cancer angiogenesis.

In one embodiment, the collagen modifying enzyme is associated with one or more of scarcoma metastasis, lung metastasis, and pulmonary metastasis.

The invention provides a system for diagnosing the progression of cancer in a subject. In one embodiment, the system comprises a probe capable of detecting the expression of one or more of HIF1α and a collagen modifying enzyme in a subject.

In one embodiment, detecting the expression of the collagen modifying enzyme a subject comprises detecting expression of the collagen modifying enzyme mRNA in a subject.

In one embodiment, detecting the expression of the collagen modifying enzyme mRNA in a subject comprises detecting expression of collagen modifying enzyme the mRNA in a tumor cell or a mesenchymal cell.

In one embodiment, detecting the expression of the collagen modifying enzyme in a subject comprises detecting expression of the collagen modifying enzyme protein in a subject.

In one embodiment, detecting the expression of the collagen modifying enzyme protein in a subject comprises detecting expression of the collagen modifying enzyme protein in a tumor cell or a mesenchymal cell.

In one embodiment, the probe comprises a nucleic acid or a protein.

In one embodiment, the system further comprises a detector capable of detecting the interaction of the probe with a target associated with the collagen modifying enzyme expression.

In one embodiment, the collagen modifying enzyme is expressed in non-metastatic cells at a first amount and the collagen modifying enzyme is expressed in metastatic cells at a second amount that is greater than the first amount.

The invention also provides a method for diagnosing the progression of cancer in a subject. In one embodiment, the method comprises detecting one or more of expression of a collagen modifying enzyme in a subject and activity of a collagen modifying enzyme in a subject; and diagnosing the progression of cancer in a subject.

In one embodiment, the collagen modifying enzyme is selected from the group consisting of procollagen-lysine 5-dioxygenase 1 (PLOD1); procollagen-lysine 2-oxoglutarate 5-dioxygenase 2 (PLOD2); procollagen-lysine 5-dioxygenase 3 (PLOD3) and any combination thereof.

The invention also provides a method for treating a neoplastic disease comprising administering to a subject an effective amount of a composition comprising an inhibitor of one or more of HIF1α and a collagen modifying enzyme.

In one embodiment, the method further comprises at least one of reducing the metastasis of a cancer in a subject, reducing the cell growth of a cancer in a subject, reducing the invasiveness of a cancer in a subject, or reducing the angiogenesis of a cancer in a subject.

In one embodiment, the neoplastic disease is a sarcoma.

In one embodiment, the inhibitor is minoxidil or a salt or chemical analog thereof.

In one embodiment, the method further comprises administering a therapeutic agent to the subject.

In one embodiment, the subject is a human.

The invention also provides a method for preventing a neoplastic disease from metastasizing. In one embodiment, the method comprises administering to a subject an effective amount of a composition comprising an inhibitor of a collagen modifying enzyme.

In one embodiment, the neoplastic disease is a sarcoma.

In one embodiment, the inhibitor is minoxidil or a salt or chemical analog thereof.

In one embodiment, the method further comprises administering a therapeutic agent to the subject.

In one embodiment, the subject is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A through 1L, is a series of imaged demonstrating that HIF1α is an important regulator of metastasis in an autochthonous, genetic model of UPS potentially via its modulation of PLOD2. (FIG. 1A) Relative gene expression in human metastatic (N=5) and non-metastatic (N=8) UPS and fibrosarcoma. HIF1α (P=0.0312) and PLOD2 (P=0.0011) were significantly upregulated in metastastic sarcomas. (FIG. 1B) Mouse models of sarcoma. LSL-KrasG12D/+;Trp53fl/fl(KP) and LSLKrasG12D/+; Trp53fl/fl;HIF1αfl/fl (KPH) genotyping showed effective recombination of HIF1αfl alleles in Adeno-Cre initiated tumors. (FIG. 1C) Mice remained tumor free for roughly 40 days, by 90 days all of the mice had developed palpable tumors (volume=200 mm3) KP; n=30, KPH; n=20; (P=0.3755). (FIG. 1D) Primary tumor size. Two weeks after tumors were palpable they had grown 2-8 fold larger, but there was no difference between KP; n=7 and KPH; n=9 tumor growth (P=0.7342) (FIG. 1E) Metastasis free survival in KP; n=33 and KPH; n=28 mice; P=0.0456. Lung metastases were confirmed histologically. (FIG. 1F) Masson's Trichrome and Picrosirius red staining of tumor nest areas and blood vessels in primary KP and KPH tumors. Deletion of HIF1α alters collagen in KPH tumors. Masson's Trichrome stains collagen fibers blue. Cells were counterstained in red with Weigert's hematoxylin. Scale bar=50 μm. (FIG. 1G) Western blot analyses of sarcoma cells derived from KP and KPH tumors. Expression of HIF1α and PLOD2 proteins is hypoxia inducible and is abolished when HIF1α is deleted. (FIG. 1H) qRT-PCR analysis of 2 individually derived KP and KPH cell lines. PLOD2 mRNA transcription is induced under hypoxic conditions in control cells (KP1; P=0.0284 and KP2; P=0.0391). Deletion of HIF1α abolished hypoxia-induced PLOD2 mRNA levels. (FIG. 1I) Western blot of PLOD2 expression in KIA cells and (FIG. 1J) HT-1080 cells. (FIG. 1K) qRT-PCR analyses of KIA cells. Expression of HIF1α and PLOD2 is hypoxia inducible (PLOD2 qRT-PCR: P=0.0284) and is abolished when HIF1α is deleted (PLOD2 qRT-PCR: P=0.0403). (FIG. 1L) HT-1080 cells were evaluated by qRT-PCR as in (FIG. 1K). Expression of HIF1α and PLOD2 is hypoxia inducible (PLOD2 qRT-PCR: P=0.0006) and is abolished when HIF1α is deleted (PLOD2 qRT-PCR: P=0.0210).

FIG. 2, comprising FIGS. 2A through 2H, is a series of images demonstrating that HIF1α and PLOD2 are dispensable for primary sarcoma formation but essential for metastasis. (FIG. 2A) Tumor allograft using 1×106 Scr, HIF1α-deficient, or PLOD2-deficient KIA cells subcutaneously injected into flanks of nude mice. n=5 mice per group, 2 tumors per mouse, i.e. 10 tumors per shRNA treatment. (B) Tumor weight was determined upon dissection of euthanized animals. PLOD2-deficient tumors were slightly larger than Scr tumors (P=0.0495) and HIF1α-deficient tumors (P=0.0131). (FIG. 2C) Lungs from mice bearing KIA transplanted subcutaneous tumors. H&E staining revealed the presence of numerous metastases in control tumors (large purple areas) but very few in lungs from animals bearing PLOD2- and HIF1α-deficient tumors. (FIG. 2D) % Tumor burden was evaluated with ImagePro7 software (FIG. 2E). Loss of HIF1α in the primary tumor significantly reduced the total number of sarcoma foci in lungs (P<0.0001) and the average number of sarcoma foci/lung (P=0.0500). Loss of PLOD2 also significantly reduced the total number of sarcoma foci in lungs (P<0.0001) and the average number of sarcoma foci/lung (P=0.0453). HIF1α and PLOD2 depletion in primary tumors also decreased the % of lungs with sarcoma foci. (FIG. 2F) H&E, HIF1α, Hypoxyprobe, and Lectin staining were used to characterize control KIA tumors using a 50× objective (scale bar; 200 μm) and a 200× objective (scale bar; 100 μm). Black boxes indicate enlarged areas; arrow (left panel) indicates HIF1α-positive cells. Arrow (right panel) indicates lectin-positive cells of a blood vessel. (FIG. 2G) Picrosiruis stain was used to characterize collagen organization in subcutaneous KIA tumors. Deletion of HIF1α and PLOD2 altered collagen organization. Scale bar=50 μm. (FIG. 2H) Hydroxyproline modifications on collagen were measured using acid hydrolyzed tumor tissue. Deletion of HIF1α (P=0.0536) and PLOD2 (P=0.0102) increased hydroxyproline levels compared with scramble control.

FIG. 3, comprising FIGS. 3A through 3D, is a series of images demonstrating that HIF1α and PLOD2 mediate sarcoma cell migration via a cell extrinsic mechanism. (FIG. 3A) Scratch migration assays of confluent KIA cells stably expressing Scr, HIF1α, or PLOD2 specific shRNAs and either copGFP (HIF1α, PLOD2) or dsRed (Scr). Cells were mixed 1:1. (FIG. 3B) Quantification of recovery in (FIG. 3A) (all P values are ≦0.0105). (FIG. 3C) Western blot analyses of KIA cells treated as in (FIG. 3A) and (FIG. 3B). ShRNA-mediated knockdown of HIF1α and PLOD2 shown here also reflects knockdown occurring in panels A and B as cell lines generated for these assays were then transduced with copGFP lentivirus of dsRed lentivirus. (FIG. 3D) Proliferation of KIA cells expressing Scr, HIF1α, or PLOD2 specific shRNAs under hypoxic conditions. Cells were counted daily.

FIG. 4, comprising FIGS. 4A through 4F, is a series of images demonstrating that PLOD2 expression rescues sarcoma migration but not invasion. (FIG. 4A) Quantification of migration assays of normoxic and hypoxic KIA cells expressing Scr or HIF1α specific shRNAs and ectopically expressing control or wild type human PLOD2 cDNA. All P values are ≦0.0432) (B) Quantification of HT-1080 migration assays performed as in (FIG. 4A) except murine Plod2 was ectopically expressed. All P values are ≦0.0431. (FIG. 4C) Representative images of boyden chamber migration assay using HT-1080 cells treated as in (FIG. 4B). (scale bar; 50 μm). (FIG. 4D) Quantification of invasion assays of normoxic and hypoxic KIA cells expressing Scr or HIF1α specific shRNAs and ectopically expressing control or wild type human PLOD2 cDNA using matrigel coated transwell invasion chambers. All P values are ≦0.0084. (FIG. 4E) Quantification of HT-1080 invasion assays performed as in (FIG. 4D) except murine Plod2 was ectopically expressed. All P values are ≦0.0017. (FIG. 4F) Representative images of matirgel coated chamber invasion assay using KIA cells treated as in (FIG. 4D) (scale bar; 50 μm).

FIG. 5, comprising FIGS. 5A through 5I, is a series of images demonstrating that PLOD2 control of cell migration and metastasis is dependent upon its lysyl hydroxylase activity. (FIG. 5A) Quantification of migration assay of normoxic and hypoxic KIA cells expressing Scr or HIF1α specific shRNAs and ectopically expressing control or mutant human PLOD2 D668A cDNA (all P values <0.0001). (FIG. 5B) Quantification of migration assay of normoxic and hypoxic HT-1080 cells expressing Scr or HIF1α specific shRNAs and ectopically expressing control or mutant murine Plod2 D689A cDNA (all P values ≦0.0106). (FIG. 5C) Scratch migration assays of HT-1080 cells stably expressing copGFP in the presence or absence of 0.5 mM Minoxidil pretreatment for 48 hrs. (FIG. 5D) Quantification of recovery from (FIG. 5C) (P values are ≦0.0058). (FIG. 5E) Western blot analyses of HT-1080 and KIA cells treated as in (FIG. 5C, 5D). (FIG. 5F) Tumor allograft growth using 1×106 KIA cells subcutaneously injected into flanks of nude mice. n=10 mice per group, 2 tumors per mouse, i.e. 20 tumors per treatment with vehicle or Minoxidil. (FIG. 5G) Lungs from mice bearing KIA transplanted subcutaneous tumors treated with PBS or Minoxidil. H&E staining revealed the presence of numerous metastases in control tumors (large purple areas) but very few in lungs from animals treated with Minoxidil. (FIG. 5H) Intra-peritoneal Minoxidil treatment reduced the average number of sarcoma foci/lung. (FIG. 5I) Picrosirius red staining of KIA subcutaneous tumors from (FIG. 5F). Minoxidil treatment altered collagen organization in the primary tumors. Scalebar=50 μm.

FIG. 6, comprising FIGS. 6A through 6E, is a series of images demonstrating that sarcoma cell migration, access to vasculature, and metastasis are dependent on HIF1α/PLOD2-mediated production of disorganized collagen in vivo. (FIG. 6A) Masson's Trichrome staining and SHG of collagen in Scr and HIF1α-deficient KIA tumors. Images of various tumor areas were taken including, areas of significant collagen deposition (collagen deposit). Scale bars for 50× images represent 200 μm and scale bars for 400× images represent 50 μm. SHG: collagen; tumor cells. Arrows indicated areas where tumor cells are elongated and adhere to collagen fibers. (FIG. 6B) Masson's Trichrome staining and SHG of collagen in Scr and HIF1α-deficient KIA tumors. Images show areas lacking large amounts of collagen (tumor nest), and tumor vasculature (blood vessel). (FIG. 6C) Quantification of collagen deposition in Scr and HIF1α-deficient KIA tumors. ImagePro7software subtracted red hues from images, leaving only blue (collagen) stain to be measured. Collagen to be quantified is outlined, and ImagePro7 software calculated these areas P<0.0001 (lower left panel). Number of collagen intersects/blood vessel was counted manually from 12 images and 4 separate primary tumors P=0.0016. (FIG. 6D) Masson's Trichrome staining and SHG of collagen in Scr and PLOD2-deficient KIA tumors. Scale bars for 50× images represent 200 μm and scale bars for 400× images represent 50 μm. Arrows indicated areas where tumor cells are elongated and adhere to collagen fibers. (FIG. 6E) Masson's Trichrome staining of control and Minoxidil treated KIA tumors. Arrow indicates the presence of collagen and tumor cells in the vasculature. Scale bars for top row 50× images represent 200 μm and scale bars for remaining row 400× images represent 50 μm.

FIG. 7, comprising FIGS. 7A through 7D, is a series of images demonstrating that expression of PLOD2 restores metastasis in animals bearing HIF1α-deficient sarcomas. (FIG. 7A) Tumor volume from Scr, and HIF1α-deficient, as well as HIF1α-deficient tumors that stably express the wild-type PLOD2 expression vector (rescue) n=6 mice per group, 2 tumors per mouse; i.e. 12 tumors per shRNA treatment. (FIG. 7B) H&E staining of lungs from Scr, HIF1α-deficient, and rescue tumor groups (HIF1α shRNA+PLOD2). Metastases are stained dark purple. (C) Quantification of lung metastases from tumors. (left panel) Total number of sarcoma foci in lungs from HIF1α-deficient tumors is decreased compared to Scr (P=0.0083) and to HIF1α+PLOD2 cDNA (P=0.0199). (right panel) Average number of sarcoma foci/lung is decreased in HIF1α-deficient tumors P=0.0132. (bottom panel) % of total lungs containing sarcoma foci in all three groups. (FIG. 7D) Model of hypoxia-dependent effects on collagen and metastasis in sarcomas.

FIG. 8, comprising FIGS. 8A through 8F, is a series of images demonstrating that HIF1α is not required for primary sarcoma formation. (FIG. 8A) (left) Tumor transplant using 1×106 Scr or HIF1α-deficient KIA cells subcutaneously injected into flanks of nude mice. (right) Tumor weight was determined upon dissection of euthanized animals. (FIG. 8B) Western blot analysis of KIA cells stably expressing Scr, HIF1α, or HIF2α shRNAs showing efficacy and specificity of HIF knockdown. (FIG. 8C) qRT-PCR of Scr and HIF1α-deficient KIA tumor tissue. (FIG. 8D) (left) Tumor transplant using 1×106 Scr or HIF1α-deficient HT-1080 cells subcutaneously injected into flanks of nude mice. (right) Tumor weight was determined upon dissection of euthanized animals. (FIG. 8E) Western blot analysis of HT-1080 cells stably expressing Scr, HIF1α, or HIF2α shRNAs. (FIG. 8F) qRT-PCR of KIA cells stably expressing Scr or HIF1α shRNAs under normoxic and hypoxic conditions.

FIG. 9, comprising FIGS. 9A through 9C, is a series of images demonstrating that HIF1α and PLOD2 mediate sarcoma cell migration. (FIG. 9A) (left) Transwell migration assay of normoxic and hypoxic KP cells expressing Scr or HIF1α specific shRNAs. Quantification showed an increase in control cell migration when cells are exposed to hypoxia (P<0.0001). The hypoxia-induced migration is lost when HIF1α is depleted (P<0.0001). (right) Transwell migration assay of normoxic and hypoxic KP cells expressing Scr or PLOD2 specific shRNAs. Quantification showed an increase in control cell migration when cells are exposed to hypoxia (P<0.0001). The hypoxia-induced migration is lost when PLOD2. (FIG. 9B) (left) Transwell migration assay of normoxic and hypoxic KIA cells expressing Scr or HIF1α specific shRNAs. Quantification showed an increase in control cell migration when cells are exposed to hypoxia (P<0.0001). The hypoxia-induced migration is lost when HIF1α is depleted (P=0.0002). (right) Transwell migration assay of normoxic and hypoxic KIA cells expressing Scr or PLOD2 specific shRNAs. Quantification showed an increase in control cell migration when cells are exposed to hypoxia (P=0.0005). The hypoxia-induced migration is lost when HIF1α is depleted (P=0.0003). (FIG. 9C) (left) Transwell migration assay of normoxic and hypoxic HT-1080 cells expressing Scr or HIF1α specific shRNAs. Quantification showed an increase in control cell migration when cells are exposed to hypoxia (P<0.0001). The hypoxia-induced migration is lost when HIF1α is depleted (P<0.0001). (right) Transwell migration assay of normoxic and hypoxic HT-1080 cells expressing Scr or PLOD2 specific shRNAs. Quantification showed an increase in control cell migration when cells are exposed to hypoxia (P<0.0001). The hypoxia-induced migration is lost when PLOD2.

FIG. 10, comprising FIGS. 10A through 10C, is a series of images demonstrating that HIF1α and PLOD2 mediate migration via a cell extrinsic mechanism in KP cells. (FIG. 10A) Scratch migration assays of confluent KP cells stably expressing Scr, HIF1α, or PLOD2 specific shRNAs and either copGFP (HIF1α, PLOD2) or dsRed (Scr). Cells are mixed 1:1. (FIG. 10B) Quantification of recovery from (FIG. 10A) (all P values are ≦0.0012). (FIG. 10C) Western blot analyses of KP cells treated as in (FIG. 10A) and (FIG. 10B). ShRNA-mediated knockdown of HIF1α and PLOD2 shown here also reflects knockdown occurring in panels FIG. 9 as cell lines generated for these assays were then transduced with copGFP lentivirus of dsRed lentivirus.

FIG. 11, comprising FIGS. 11A through 11D, is a series of images demonstrating that HIF1α and PLOD2 mediate migration via a cell extrinsic mechanism in HT-1080 cells. (FIG. 11A) Scratch migration assays of confluent HT-1080 cells stably expressing Scr, HIF1α, or PLOD2 specific shRNAs and either copGFP (HIF1α, PLOD2) or dsRed (Scr). Cells are mixed 1:1. (FIG. 11B) Quantification of recovery from (FIG. 11A) (all P values are ≦0.0123). (FIG. 11C) Western blot analyses of HT-1080 cells treated as in (FIG. 11A) and (FIG. 11B). ShRNA-mediated knockdown of HIF1α and PLOD2 shown here also reflects knockdown occurring in FIG. 9 as cell lines generated for these assays were then transduced with copGFP lentivirus of dsRed lentivirus. (FIG. 11D) Proliferation of HT-1080 cells expressing Scr, HIF1α, or PLOD2 specific shRNAs under hypoxic conditions. Cells were counted daily.

FIG. 12, comprising FIGS. 12A through 12E, is a series of images demonstrating that expression of WT and mutant PLOD2 in sarcoma cells. (FIG. 12A) Western blot analyses of PLOD2 and HIF1α in HT-1080 cells treated as in FIG. 4B, 4C. Wild type PLOD2; upper band, ectopically expressed PLOD2; lower band. (FIG. 12B) qRT-PCR analyses of KIA cells treated as in FIG. 4A. (FIG. 12C) qRT-PCR analyses of KIA cells treated as in FIG. 5B. (FIG. 12D) qRT-PCR analyses of HT-1080 cells treated as in FIG. 5B. (FIG. 12E) Western blot analyses of HT-1080 and KIA cells treated as in FIG. 5A, 5B.

FIG. 13, comprising FIGS. 13A and 13B, is a series of images demonstrating that Minoxidil treatment does not affect primary tumor volume or overall animal weight. (FIG. 13A) Tumor weight was determined upon dissection of euthanized animals. Minoxidil treatment had no effect on primary KIA tumor volume or mass. (FIG. 13B) Animals were weighed every other day and their overall health evaluated. Animal weight and health were unaffected by Minoxidil treatment.

FIG. 14, comprising FIGS. 14A and 14B, is a series of images demonstrating that tumor cells are the major source of sarcoma collagen in vivo. (FIG. 14A) KIA tumors contain low levels of infiltrating cells. Immunofluorescence staining of KIA subcutaneous tumor sections using antibodies to GFP and the mesenchymal marker, Vimentin. GFP negative; Vimentin positive cells are indicative of the infiltrating cell population. (FIG. 14B) Flow cytometry of dissociated GFP positive tumors. ˜12% of KIA tumors consisted of GFP negative cells.

### DETAILED DESCRIPTION

The present invention is directed to methods and compositions for treatment, inhibition, prevention, or reduction of metastasis in cancers. In particular, the invention is related to compositions and methods affecting one or more of the level, production, and activity of a collagen modifying enzyme. In one embodiment, the collagen modifying enzyme exhibits a lysyl hydroxylase activity. In another embodiment, the collagen modifying enzyme includes but is not limited to procollagen-lysine 5-dioxygenase 1 (PLOD1), procollagen-lysine, 2-oxoglutarate 5-dioxygenase 2 (PLOD2), procollagen-lysine 5-dioxygenase 3 (PLOD3).

An aspect of the present invention comprises a method for interfering with the activity of a collagen modifying enzyme comprising administering to a subject an effective amount of a composition comprising an inhibitor of the collagen modifying enzyme (e.g., PLOD2). In an embodiment of the present invention, the composition prevents the transcription of collagen modifying enzyme genes or translation of collagen modifying enzyme mRNA. In another embodiment of the present invention, the composition interferes with the activity of a collagen modifying enzyme activity. The composition that interferes with the activity can comprise an antibody or a fragment thereof that binds to at least a portion of the collagen modifying enzyme, a peptide, a nucleic acid, or small molecule.

In one embodiment, a method for interfering with the activity of a collagen modifying enzyme (e.g., PLOD2) can comprise interfering with the activity of the collagen modifying enzyme associated with cancer metastasis. In another embodiment of the present invention, a method for interfering with the activity of a collagen modifying enzyme comprises interfering with the activity of the collagen modifying enzyme associated with sarcoma cell metastasis. In yet an embodiment of the present invention, a method for interfering with the activity of the collagen modifying enzyme associated with pulmonary metastases.

Another aspect of the present invention comprises a pharmaceutical composition comprising an inhibitor of a collagen modifying enzyme (e.g., PLOD2). In an embodiment of the present invention, the inhibitor of a collagen modifying enzyme can compromise an antibody or a fragment thereof that binds to at least a portion of the a collagen modifying enzyme, a peptide, a nucleic acid, or small molecule and is capable of at least one of interfering with the activity of the a collagen modifying enzyme; preventing the transcription of a collagen modifying enzyme genes; or translation of a collagen modifying enzyme mRNA.

In one embodiment, the method and composition for treatment, inhibition, prevention, or reduction of metastasis in cancers comprises a pharmaceutical composition containing minoxidil or a salt or chemical analog thereof.

The invention also provides compositions and methods to inhibit one or more of HIF1α and a collagen modifying enzyme. In one embodiment, the inhibitor of the invention can be used in combination with another therapeutic agent.

### DEFINITIONS

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or in some instances±10%, or in some instances±5%, or in some instances±1%, or in some instances±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A disease or disorder is “alleviated” if the severity of a sign or symptom of the disease or disorder, the frequency with which such a sign or symptom is experienced by a patient, or both, is reduced. “Alleviating” specific cancers and/or their pathology includes degrading a tumor, for example, breaking down the structural integrity or connective tissue of a tumor, such that the tumor size is reduced when compared to the tumor size before treatment. “Alleviating” metastasis of cancer includes reducing the rate at which the cancer spreads to other organs.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The an antibody in the present invention may exist in a variety of forms where the antigen binding portion of the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to at least one portion of an intact antibody and refers to the antigenic determining variable regions of an intact antibody. Examples of antibody fragments include, but are not limited to, Fab, Fab′, F(ab′)2, and Fv fragments, linear antibodies, sdAb (either V_{L} or V_{H}), camelid V_{HH} domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

An “antibody heavy chain,” as used herein, refers to the larger of the two types of polypeptide chains present in antibody molecules in their naturally occurring conformations, and which normally determines the class to which the antibody belongs.

An “antibody light chain,” as used herein, refers to the smaller of the two types of polypeptide chains present in antibody molecules in their naturally occurring conformations. Kappy (K) and lambda (λ) light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “cancer” as used herein is defined as disease characterized by the abnormal growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, sarcoma and the like.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., DNA, cDNA, rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “inhibit,” as used herein, means to suppress or block an activity or function by at least about ten percent relative to a control value. Preferably, the activity is suppressed or blocked by 50% compared to a control value, more preferably by 75%, and even more preferably by 95%.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Sample” or “biological sample” as used herein means a biological material from a subject, including but is not limited to organ, tissue, exosome, blood, plasma, saliva, urine and other body fluid. A sample can be any source of material obtained from a subject.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

The term “therapeutic” as used herein means a treatment and/or prophylaxis. A therapeutic effect is obtained by suppression, remission, or eradication of a disease state.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based on the discovery that hypoxia controls sarcoma cell metastasis through a novel mechanism in which HIF1α induces the expression of the intracellular enzyme procollagen-lysine 2-oxoglutarate 5-dioxygenase 2 (PLOD2). The invention relates to the discovery that loss of HIF1α or PLOD2 expression disrupts collagen modification, cell migration and pulmonary metastasis. Accordingly, the invention provides compositions and methods for targeting one or more of HIF1α and PLOD2 as a novel therapeutic target for preventing metastasis of cancer.

Another aspect of the invention provides agents and compositions for use in the treatment of cancer metastasis. One aspect of the invention provides a composition for use in inhibiting metastasis comprising a collagen modifying enzyme antagonist. An antagonist includes but is not limited to a molecule which blocks or reduces the expression or biological activity of a collagen modifying enzyme gene product. Antagonists may include proteins, nucleic acids, carbohydrates, or any other molecules which bind or interact with the collagen modifying enzyme gene product (e.g., PLOD2).

In one embodiment, the method and composition for treatment, inhibition, prevention, or reduction of metastasis in cancers comprises a pharmaceutical composition containing minoxidil or a salt or chemical analog thereof.

### Compositions

In one embodiment, the invention provides an inhibitor of one or more of HIF1α and a collagen modifying enzyme. In various embodiments, the present invention includes compositions for inhibiting the level or activity of a collagen modifying enzyme such as PLOD2 in a subject, a tissue, or an organ in need thereof. In various embodiments, the compositions of the invention decrease the amount of polypeptide of the desired collagen modifying enzyme, the amount of mRNA of the collagen modifying enzyme, the amount of enzymatic activity of the collagen modifying enzyme, or a combination thereof.

It will be understood by one skilled in the art, based upon the disclosure provided herein, that a decrease in the level of the collagen modifying enzyme encompasses the decrease in the expression, including transcription, translation, or both. The skilled artisan will also appreciate, once armed with the teachings of the present invention, that a decrease in the level of the collagen modifying enzyme includes a decrease in the activity of the enzyme. Thus, decrease in the level or activity of the collagen modifying enzyme includes, but is not limited to, decreasing the amount of polypeptide of the collagen modifying enzyme, and decreasing transcription, translation, or both, of a nucleic acid encoding the collagen modifying enzyme; and it also includes decreasing any activity of the collagen modifying enzyme as well.

In one embodiment, the invention provides a generic concept for inhibiting the collagen modifying enzyme as an anti-tumor therapy. In one embodiment, the composition of the invention comprises an inhibitor of the collagen modifying enzyme. In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

One skilled in the art will appreciate, based on the disclosure provided herein, that one way to decrease the mRNA and/or protein levels of one or more of the collagen modifying enzyme in a cell is by reducing or inhibiting expression of the nucleic acid encoding the collagen modifying enzyme. Thus, the protein level of the desired collagen modifying enzyme in a cell can also be decreased using a molecule or compound that inhibits or reduces gene expression such as, for example, siRNA, an antisense molecule or a ribozyme.

siRNA

In one embodiment, siRNA is used to decrease the level of one or more of HIF1α and a collagen modifying enzyme (e.g., PLOD2). RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of the desired collagen modifying enzyme at the protein level using RNAi technology.

In other related aspects, the invention includes an isolated nucleic acid encoding an inhibitor, wherein an inhibitor such as an siRNA or antisense molecule, inhibits the desired collagen modifying enzyme, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein. In another aspect of the invention, the desired collagen modifying enzyme, or a regulator thereof, can be inhibited by way of inactivating and/or sequestering one or more of the collagen modifying enzyme, or a regulator thereof. As such, inhibiting the effects of the collagen modifying enzyme can be accomplished by using a transdominant negative mutant.

In another aspect, the invention includes a vector comprising an siRNA or antisense polynucleotide. Preferably, the siRNA or antisense polynucleotide is capable of inhibiting the expression of the desired collagen modifying enzyme. The incorporation of a desired polynucleotide into a vector and the choice of vectors is well-known in the art as described in, for example, Sambrook et al., supra, and Ausubel et al., supra, and elsewhere herein.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Antisense Nucleic Acids

In one embodiment of the invention, an antisense nucleic acid sequence which is expressed by a plasmid vector is used to inhibit a desired collagen modifying enzyme. The antisense expressing vector is used to transfect a mammalian cell or the mammal itself, thereby causing reduced endogenous expression of the collagen modifying enzyme.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Compositions and methods for the synthesis and expression of antisense nucleic acids are as described elsewhere herein.

Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit a desired collagen modifying enzyme. Ribozymes useful for inhibiting the expression of a target molecule may be designed by incorporating target sequences into the basic ribozyme structure which are complementary, for example, to the mRNA sequence of the collagen modifying enzyme of the present invention. Ribozymes targeting a desired collagen modifying enzyme may be synthesized using commercially available reagents (Applied Biosystems, Inc., Foster City, Calif.) or they may be genetically expressed from DNA encoding them.

Small Molecules

When the inhibitor of the invention is a small molecule, a small molecule agonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In one embodiment, the small molecule is able to inhibit one or more of HIF1α and a collagen modifying enzyme. In one embodiment, the small molecule induces the expression of the intracellular enzyme procollagen-lysine, 2-oxoglutarate 5-dioxygenase 2 (PLOD2). In one embodiment, the small molecule is minoxidil or a salt or chemical analog thereof.

Antagonist

In another aspect of the invention, one or more of HIF1α and a collagen modifying enzyme can be inhibited by way of inactivating and/or sequestering the collagen modifying enzyme. As such, inhibiting the effects of a collagen modifying enzyme can be accomplished by using a transdominant negative mutant. Alternatively an antibody specific for the collagen modifying enzyme, otherwise known as an antagonist to the collagen modifying enzyme may be used. In one embodiment, the antagonist is a protein and/or compound having the desirable property of interacting with a binding partner of the collagen modifying enzyme and thereby competing with the corresponding protein. In another embodiment, the antagonist is a protein and/or compound having the desirable property of interacting with the collagen modifying enzyme and thereby sequestering the collagen modifying enzyme.

As will be understood by one skilled in the art, any antibody that can recognize and bind to an antigen of interest is useful in the present invention. Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magenetic-actived cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

### Methods

The invention provides methods of treating or preventing cancer, or of treating and preventing metastasis of tumors. Related aspects of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective metastasis-inhibiting amount of an inhibitor of one or more of HIF1α and a collagen modifying enzyme. The invention further provides a method of inhibiting metastasis in an individual in need thereof, the method comprising administering to the individual an effective metastasis-inhibiting amount of any one of the compositions described herein.

In some embodiments of the methods for inhibiting metastasis in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The disclosed compounds can be used to prevent, abate, minimize, control, and/or lessen tumor metastasis in humans and animals. The disclosed compounds can also be used to slow the rate of primary tumor growth. The disclosed compounds when administered to a subject in need of treatment can be used to stop the spread of cancer cells. As such, the compounds disclosed herein can be administered as part of a combination therapy with one or more drugs or other pharmaceutical agents. When used as part of the combination therapy, the decrease in metastasis and reduction in primary tumor growth afforded by the disclosed compounds allows for a more effective and efficient use of any pharmaceutical or drug therapy being used to treat the patient. In addition, control of metastasis by the disclosed compound affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of one or more of the disclosed compounds to a subject diagnosed with a malignant tumor or cancerous cells or to a subject having a tumor or cancerous cells.

The following are non-limiting examples of cancers that can be treated by the disclosed methods and compositions: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

In one embodiment, the invention provides a method to treat cancer metastasis comprising treating the subject prior to, concurrently with, or subsequently to the treatment with an inhibitor of one or more of HIF1α and a collagen modifying enzyme of the invention, with a complementary therapy for the cancer, such as surgery, chemotherapy, chemotherapeutic agent, radiation therapy, or hormonal therapy or a combination thereof.

In another embodiment, the invention provides a method to treat cancer metastasis comprising treating the subject prior to, concurrently with, or subsequently to the treatment with minoxidil or a salt or chemical analog thereof, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### Dosage and Formulation (Pharmaceutical Compositions)

The present invention envisions treating a disease, for example, cancer and the like, in a mammal by the administration of therapeutic agent, e.g. an inhibitor of a collagen modifying enzyme.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent or modified cell may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The agents of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Hypoxia-Dependent Modification of Collagen Networks Promotes Sarcoma Metastasis

The development of successful therapeutic interventions for sarcoma depends on the ability to accurately model UPS and other sarcomas. One mouse model for investigating UPS employs simultaneous Cre-dependent expression of oncogenic Kras^{G12D} and deletion of p53 in the left gastrocnemius muscle (Kirsch et al., 2007, Nat Med 13(8):992-7). These genetic changes occur frequently in sarcoma and the murine tumors that develop recapitulate human UPS morphologically, histologically, and genetically (Kirsch et al., 2007, Nat Med 13(8):992-7; Mito et al., 2009, PLoS One 4(11):e8075). Most importantly, primary tumors that develop in this autochthonous model successfully metastasize to the lung, mirroring human UPS. Furthermore, subcutaneous allografts of murine UPS cells also metastasize to the lung within several weeks of implantation. Combined, these approaches allow for the investigation of molecular mechanisms that govern primary UPS formation and pulmonary metastases.

Intratumoral hypoxia and expression of Hypoxia Inducible Factor1α (HIF1α) correlate with metastasis and poor survival in sarcoma patients. The results presented herein demonstrate that hypoxia controls sarcoma cell metastasis through a novel mechanism in which HIF1α induces the expression of the intracellular enzyme procollagen-lysine, 2-oxoglutarate 5-dioxygenase 2 (PLOD2). PLOD2 hydroxylates procollagen, consequently altering the organization of extracellular collagen fibers along which transformed cells migrate in primary tumors. The results show that loss of HIF1α or PLOD2 expression disrupts collagen modification, cell migration and pulmonary metastasis (but not primary tumor growth) in allograft and autochthonous LSL-Kras^{G12D/+}; Trp53^{fl/fl} murine sarcoma models. Furthermore, ectopic PLOD2 expression restores migration and metastatic potential in HIF1α-deficient cells and tumors, and microarray analyses of human sarcomas revealed elevated HIF1α and PLOD2 expression in metastatic primary lesions. Importantly, pharmacological inhibition of PLOD enzymatic activity suppressed pulmonary metastases. Collectively, these data indicate that HIF1α controls sarcoma metastasis through PLOD2-dependent collagen modification and organization in primary tumors. Accordingly, the invention provides compositions and methods for targeting PLOD2 as a therapeutic in sarcomas and successful inhibition of this enzyme reduces tumor cell dissemination.

The materials and methods employed in this example are now described.

### Material and Methods

Microarray Analysis

RNA was isolated from human tumor tissue using RNeasy kit (Qiagen) and quality was analyzed using a 2100 Bioanalyzer (Agilent Technologies). Amplification was achieved using the TotalPrep RNA Amplification Kits (Illumina). Amplified cRNAs were then hybridized on HumanRef8 Expression Beadchips (Illumina), which target more than 24,000 genes. Image analysis was performed using Illumina's BeadStudio v3.0.14 Gene Expression Module. All statistical analyses were done using the statistical software R (http://www.r-project.org). Supervised hierarchical clustering of 140 genes transcriptionally regulated by HIF1α was performed using 1-r (Pearson correlation) as a distance metric with a complete linkage. Data from this analysis was used to determine relative gene enrichment.

### Mouse Models

Generation of Hif1α^{fl/fl} mice (Ryan et al., 2000, Cancer Res 60(15):4010-5) and LSL-Kras^{G12D/+}Trp53^{fl/fl} mice has been previously described (Kirsch et al., 2007, Nat Med 13(8):992-7). These mice were crossed to create the LSL-Kras^{G12D/+}Trp53^{fl/fl}Hif1a^{fl/fl} animals. Soft tissue sarcomas were generated by intramuscular injection of a calcium phosphate precipitate of Ad-Cre (Gene Transfer Vector Core, University of Iowa). For transplant tumors 1×106 KIA (derived from LSL-Kras^{G12D/+}Ink4d^{fl/fl} tumors) were injected subcutaneously into the flanks of nu/nu mice (Charles River Laboratories). In each experiment 10 mice per experimental group were used with each mouse bearing two subcutaneous tumors. Tumors developed after 3-5 days, were monitored every other day, and animals were euthanized after 20-30 days. For in vivo lung metastasis analysis lung tissues were removed, and sections were stained with hematoxylin and eosin. Images were acquired using a Nikon SMZ800 stereoscope with a Nikon 1200F digital camera and Nikon Act-1 software. The ratio of total metastatic sarcoma foci to total lung area (% tumor burden) was determined using ImagePro 6.3 software (Media Cybernetics Inc.). Additionally, the percent of animals in the entire cohort with metastatic foci was evaluated along with total number of metastatic foci per experiment and the averae number of sarcoma lung foci per lung in each experiment. For Minoxidil injections, allografts and drug treatments were started simultaneously. Mice bearing subcutaneous KIA tumors on each flank were intraperitoneal injected with PBS, 1 mg/kg Minoxidil, or 3 mg/kg Minoxidil every other day unless the animals were euthanized, the tumors and lungs removed.

Cell Culture, Treatment and Lentiviral Transduction

HT-1080 cells were purchased from ATCC (Manassas, Va.). KIA cells were derived from LSL-Kras^{G12D/+}Ink4d^{fl/fl} tumors as described elsewhere (Kirsch et al., 2007, Nat Med 13(8):992-7; Yoon et al., 2009, Int J Radiat Oncol Biol Phys 74(4):1207-16). Finally, KP1 and KP2 were derived from LSL-Kras^{G12D/+}Trp53^{fl/fl} tumors and KPH1 and KPH2 cells were derived from LSL-Kras^{G12D/+}Trp53^{fl/fl}Hif1a^{fl/fl} tumors. Low-oxygen conditions were achieved in a Ruskinn in vivO₂ 400 work station. Cells were treated with 0.5 mM Minoxidil diluted in DMEM culture media (Sigma Aldrich) for 36-48 hrs, and drug was replenished every 24 hrs. For shRNA-mediated knockdown of Hif1α, HIF1α, Plod2, and PLOD2 lentiviral particles bearing pLKO.1 shRNA plasmids were generated in HEK-293T cells. 293T cells were transfected overnight with pLKO.1 empty vector, nonspecific shRNA, or target-specific shRNA and viral packaging plasmids, according to the Fugene reagent protocol (Roche). The following shRNA pLKO.1 plasmids were employed: pLKO.1 scrambled shRNA (Addgene 1864), pLKO.1 Hif1α shRNA (TRCN0000054448), pLKO.1 HIF1α shRNA (TRCN0000003808), pLKO.1 Plod2 (TRCN0000076411), PLOD2 (TRCN0000064809), G protein of vesicular stomatitis virus (VSV-G), pMDLG, pRSV-rev. Supernatant was harvested from cultures at 24 hrs and 48 hrs posttransfection, and virus concentrated using 10-kDa Amicon Ultra-15 centrifugal filter units (Millipore). Ectopic expression of wild type and mutant PLOD2 was achieved using pCDH-CMV-MCS-EF1-Puro expression vectors (System Biosciences) and were cloned in via Xbal and NHE1 restriction sites from murine or human pCMV-SPORT6 PLOD2 (Open Biosystems). PLOD2 mutants were generated using the QuikChange II site-directed mutagenesis kit (Agilent Technologies). pLKO.1 shRNA and pCDH-CMV-MCS-EF1-Puro plasmids contain a puromycin resistance gene, thus transduction efficiency was evaluated by puromycin selection. Cells were used for assays 4 days postransduction. GFP was introduced using the pCDH-CMV-EF1-copGFP vector (System Biosciences). copGFP (copepod GFP) is particularly bright and thus is suitable for in vivo SHG studies as well as cell culture. dsRED was expressed from the pULTRA-HOT vector (Addgene plasmid#24130) originally constructed by Didier Trono.

Immunostaining and Imaging

Immunohistochemistry of tissue sections with antibodies to HIF1α (Abcam) and Lectin (Vector Labs) was performed using enzymatic Avidin-Biotin Complex (ABC)-diaminobenzidine (DAB) staining (Vector Labs). Nuclei were counterstained with hematoxylin. Immunofluorescences staining of copGFP (Evrogen) and Vimentin (Abcam) as well as DAPI (Invitrogen) stained images were visualized using an Olympus IX81 microscope. Collagen was stained using Masson's Trichrome Kit (Sigma Aldrich) and nuclei were counterstained with Weigert's iron hematoxylin (Sigma Aldrich). Collagen second harmonic generation (SHG) images were captured using a Prairie Technologies Ultima 2-Photon Microscope system (Middleton, Wis.). Images were taken with an excitation wavelength of 910 nm, and captured through an emission filter of 457-487 nm (that detects the SHG signal for collagen). Collagen was quantified using Image-Pro software. All comparative images were obtained using identical microscope and camera settings. Picrosirius Red staining (Electron Microscopy Sciences) and analysis was conducted using paraffin sections of primary murine sarcomas stained with 0.1% Picrosirius and counterstained with Weigert's Hematoxylin to visualize fibrillar collagen. Sections were imaged using a Leica DMRB microscope bearing an analyzer and polarizer (leica) and an Olympus DP72 camera.

Western Blotting and qRT-PCR

Whole cell lysates were prepared in SDS/Tris pH 7.6 lysis buffer. Proteins were electrophoresed and separated by SDS-PAGE and transferred to nitrocellulose membranes and probed with the following antibodies: rabbit anti-HIF1α (Cayman Chemical co.), rabbit anti-GAPDH (Cell Signaling Inc.), and rabbit anti-PLOD2 (Proteintech). Densitometry was performed using ImageJ software. Representative western blots from multiple independent experiments are presented. Total RNA was isolated from cells using the TRIzol reagent protocol (Invitrogen) and from tumor tissue using the RNAeasy minikit (Qiagen). mRNA was reverse transcribed using the High-Capacity RNA-tocDNA kit (Applied Biosystems). Transcript expression was determined by quantitative PCR of synthesized cDNA using the Applied Biosystems 7900HT system. Target cDNA amplification was measured using TaqMan primer/probe sets (Applied Biosystems) for human and murine Hif1α, Plod2, HPRT1 (control), Lox, Serpine 2, Col5a1, and Itgav.

Migration, Invasion, and Proliferation assays

Migration assays were performed using 24-well chambers with inserts (8-μm pores) (BD Biosciences). Medium containing 10% serum was placed in the lower chamber, and tumor cells (1×10⁵) suspended in medium without serum were added to the top chamber. The plates were incubated under 21% or 0.5% 0₂ for 4 hrs (HT-1080) or 18 hrs (KIA). After migration, nonmigratory cells were removed from the top of the insert membrane using cotton swabs. The underside of each membrane was fixed in Methanol and stained with DAPI (Invitrogen), and the number of cells that migrated completely through the 8-μm pore was determined in 6 random high-power fields (20× objective) for each membrane. Invasion was examined in a similar way using Matrigel-coated inserts (BD Biosciences). Scratch assays were performed on confluent KP, KIA, and HT-1080 cells expressing either dsREd or copGFP and seed on to plates at a 1:1 ratio. Cells were imaged under normoxic conditions. ImageJ software was used to measure areas devoid of cells in 5 unique fields per condition. As cells migrated into the wounds, those areas became smaller. The average area lacking GFP+ cells per condition was determined and displayed those means as a normalized percentage with the pre-wounding image representing the baseline and then generated recovery statistics. Proliferation was assessed by counting cell numbers manually using a hemocytometer every day for 4 days.

Flow Cytometry

Tumors were dissected, homogenized and collagenase treated to generate a single cell suspension. Live cells were run on a BD LSR II flow cytometer for the detection of GFP. GFP negative parent cell lines were also run to set up GFP+ and GFP-gates.

Statistical Analysis

Data are represented as mean±SEM. Unpaired 2-tailed Student's t test was preformed for most of the studies to evaluate the differences between the control and experimental groups. P≦0.05 was considered statistically significant. Significance is indicated by the presence of an asterisk “*”. Quantified data shown represent at least 3 independent experiments. GraphPad Prism software (La Jolla, Calif.) was used to conduct all statistical analyses.

The results of the experiments are now described.

Elevated HIF1α and PLOD2 Correlate with Sarcoma Metastasis but not Primary Tumor Formation in Human and Autochthonous Murine Tumors

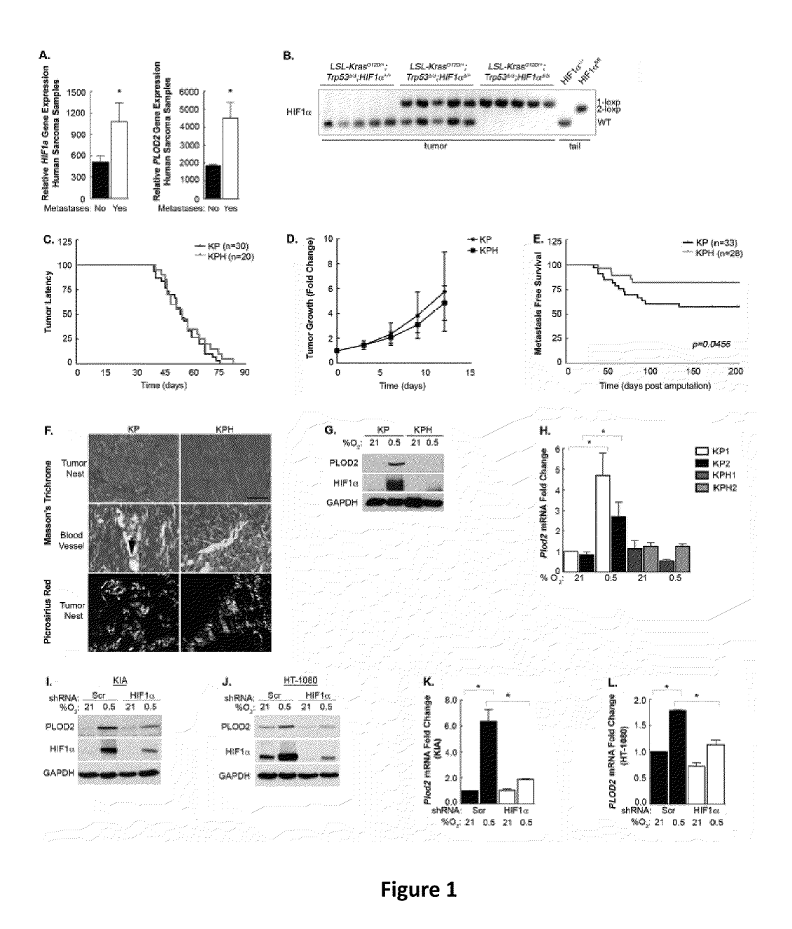

To determine if HIF1α dependent upregulation of PLOD2 could promote metastasis in primary human sarcomas, experiments were performed to compare relative gene expression based on microarray analysis of human metastatic and non-metastatic UPS and fibrosarcomas obtained prior to therapeutic intervention (Detwiller et al, 2005, Cancer Res 65(13):5881-9). HIF1α and PLOD2 expression was selectively elevated in metastatic tumors (FIG. 1A); in contrast, expression of PLOD1, a closely related isoform of PLOD2, and lysyl oxidase (LOX), another HIF1α transcriptional target and collagen-modifying enzyme, was not significantly altered (data not shown). These data suggest that HIF1α-mediated PLOD2 expression is associated with sarcoma metastasis.

Experiments employed the genetically engineered murine LSL-Kras^{G12D/+}; Trp53^{fl/fl} (KP) model of UPS (Kirsch et al., 2007, Nat Med 13(8):992-7; Mito et al., 2009, PLoS One 4(11):e8075) to investigate the effects of HIF1α and its target genes on soft tissue sarcoma development. In this model, injection of Adenovirus expressing Cre recombinase (Adeno-Cre) into the left gastrocnemius muscle resulted in Kras^{G12D} expression and Trp53 deletion, producing sarcomas within approximately 8 weeks. KP mice was also crossed with HIF1α^{fl/fl} animals to generate the LSL-Kras^{G12D/+}; Trp53^{fl/fl}; HIF1α^{fl/fl} “KPH” strain, in which HIF1α is deleted in the Kras^{G12D}-expressing, p53-deficient tumors. Genetic analysis showed highly effective Cre-dependent recombination of HIF1α^{fl/fl} alleles in the resulting sarcomas (FIG. 1B). KP and KPH animals developed tumors of similar size and latency indicating that loss of HIF1α did not alter primary tumor latency (FIG. 1C) or tumor growth (FIG. 1D). However, HIF1α deletion dramatically reduced the occurrence of pulmonary metastasis in this model, indicating that HIF1α specifically modulates tumor cell dissemination in sarcomas (FIG. 1E). Analysis of primary sarcomas by Masson's Trichrome staining of KP and KPH tumors revealed that HIF1α deletion significantly alters deposited collagen (FIG. 1F). No collagen fibers were found intersecting blood vessels in KPH tumors, whereas in KP tumors long strands of collagen with associated tumor cells were observed invading the vasculature (arrow, FIG. 1F). Picrosirius red staining revealed that HIF1α deletion has an unexpected effect on collagen organization (FIG. 1F, bottom panel). The collagen found in KPH tumors emitted red birefringence, indicating higher levels of organization, while KP tumors containing collagen emitted green birefringence that is more immature. Processed mature collagen emits red birefringence in normal tissues (Singh et al., 2012, Natl J Maxillofac Surg 3(1):15-20). However, collagen organization/maturity can be aberrant in tumors, as indicated by green birefringence (Aparna and Chant, 2010, Journal of Clinical and Diagnostic Research 4:3444-9). Furthermore, collagen organization has been shown to decrease (change birefringence gradually from red to green) as tumors worsen in stage and grade. The lack of mature collagen organization in KP tumors is consistent with the idea that normal collagen modification and processing is disrupted due to elevated HIF1α/PLOD2 activity.

Collectively, these findings suggest that the loss of HIF1α alters collagen fiber deposition in primary sarcomas, and that PLOD2 may be a critical downstream target. To quantify the levels of PLOD2 in control and HIF1α-deficient sarcomas, cell lines were derived from KP and KPH tumors. HIF1α and PLOD2 were elevated in KP cells exposed to hypoxia (0.5% O₂) for 16 hours, whereas KPH cells did not express HIF1α or PLOD2 under these conditions (FIG. 1G). Moreover, qRT-PCR showed that Plod2 mRNA levels were increased in hypoxic KP (KP1 and KP2) cells but not in KPH cells (KPH1 and KPH2) (FIG. 1H).

To demonstrate that the results were not unique to a specific genetic background, tumor cell lines derived from a distinct mouse model of sarcoma, LSL-KrasG12D/+; Ink4a/Arffl/fl “KIA” were investigated. Sarcomas initiated by Adeno-Cre injection into KIA mice display similar growth kinetics and histopathology as in KP mice. It was confirmed that PLOD2 is a hypoxia-induced HIF1α target in KIA cells (FIG. 1I). Quantification of 3 independent western blots showed that PLOD2 was significantly upregulated under hypoxia (P=0.0118) (data not shown). Deletion of HIF1α significantly abrogated hypoxia-induced PLOD2 expression (P=0.0008). Similar results were obtained using the human fibrosarcoma cell line, HT-1080 (FIG. 1J). Quantification of 3 independent western blots showed that PLOD2 was significantly upregulated under hypoxia (P=0.0301) (data not shown). Deletion of HIF1α significantly abrogated hypoxia-induced PLOD2 expression (P=0.0095). qRT-PCR analyses of KIA (FIG. 1K) and HT-1080 (FIG. 1L) cells recapitulated these observations and showed that HIF1α regulates PLOD2 at the mRNA level. It was concluded that HIF1α regulates PLOD2 expression in human and murine sarcoma cells, and alters collagen deposition in primary murine sarcomas.

### HIF1α and PLOD2 are Required for Metastasis in Sarcoma

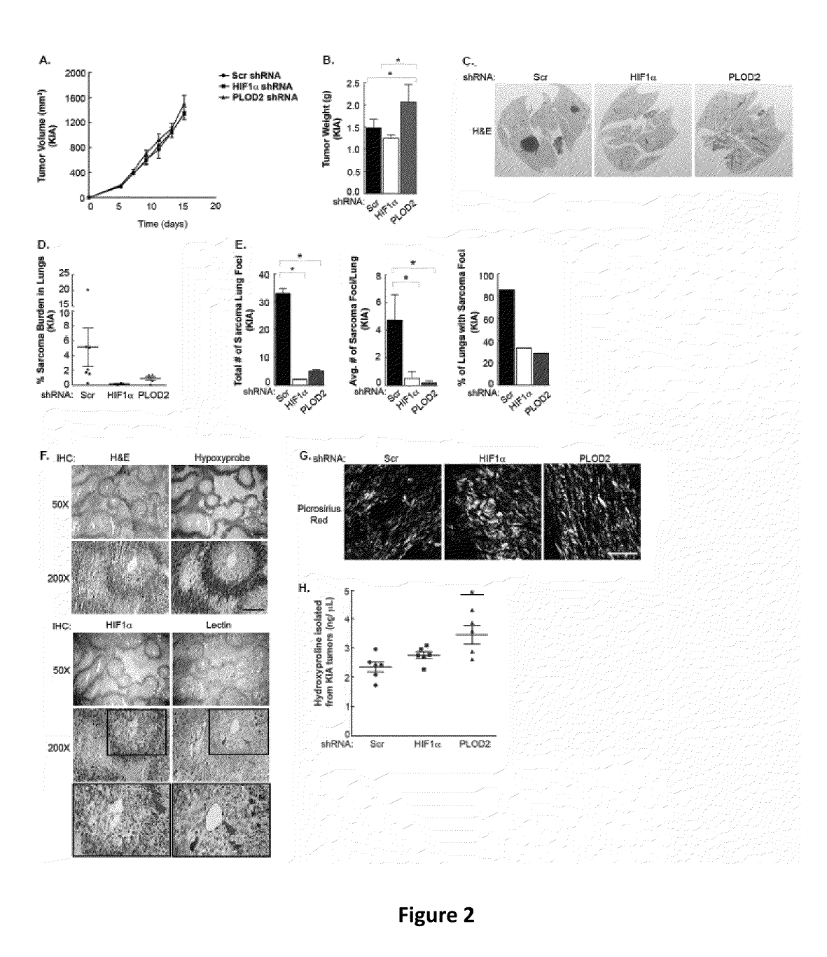

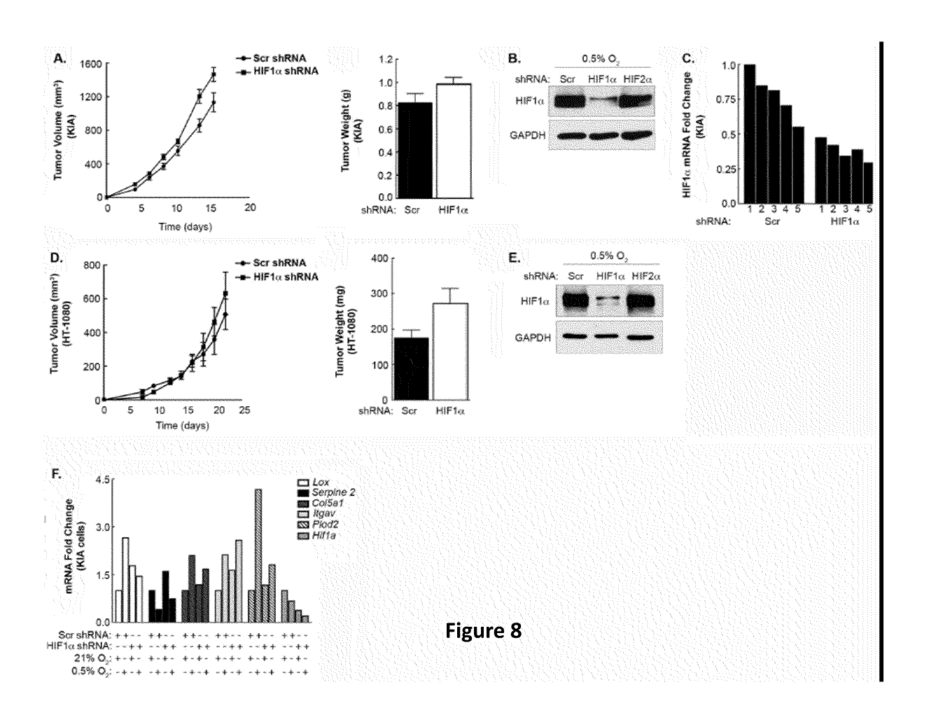

To establish a role for the HIF1α/PLOD2 pathway in sarcoma metastasis, KIA (FIG. 8A-C) and HT-1080 (FIG. 8D,E) cells transduced with lentivirus expressing Scr or HIF1α shRNA were injected subcutaneously into nude mice. No significant change was observed in primary tumor volume or weight. Subsequently, similar experiments were performed including PLOD2 shRNA in addition to Scr and HIF1α shRNA (FIG. 2A). Although the mean weight of PLOD2-deficient tumors was slightly higher than that of control or HIF1α-deficient tumors, no differences in tumor volume were observed between groups, indicating that HIF1α and PLOD2 have little effect on primary tumor volume (FIG. 2A) and weight (FIG. 2B). However, silencing HIF1α or PLOD2 caused a striking reduction in lung metastases in KIA transplanted tumors, indicating that the HIF1α/PLOD2 axis is necessary for pulmonary metastasis (FIG. 2C,D,E). HIF1α ablation did not affect the expression of related HIF1α targets, other than Plod2, including Lox, Serpine 2, Col5a1, and Itgav in HIF1α-depleted KIA tumors (data not shown) and cultured cells (FIG. 8F). It was confirmed that KIA tumors are hypoxic using Hypoxyprobe and HIF1α staining (FIG. 2F). Serial sectioning of KIA tumors showed that hypoxic regions circumscribe more oxygenated cells surrounding blood vessels, which can be visualized by Lectin staining (arrows, FIG. 2F). Picrosirius red staining of these tumor sections revealed that collagen is more organized in HIF1α/PLOD2 deleted tumors (FIG. 2G), consistent with analyses of KP and KPH primary tumors (FIG. 1F). Without wishing to be bound by any particular theory, it is believed that the changes observed in collagen organization may be due to alterations in post-translational modifications found on collagen. Deletion of HIF1α and PLOD2 results in increased hydroxyproline levels in KIA tumors (FIG. 2H), indicating that a high level of PLOD2 activity, resulting in elevated lysine hydroxylation, suppresses proline hydroxylation and mature “normal” collagen organization. Therefore, HIF1α/PLOD2 deletion allows for increased prolyl hydroxylation, stabilizing mature collagen organization. Based on these data, it was concluded that HIF1α-mediated PLOD2 expression is not essential for primary sarcoma tumor growth, but is required for efficient lung metastasis through effects on collagen maturation.

### HIF1α and PLOD2 Specifically Control Sarcoma Cell Migration

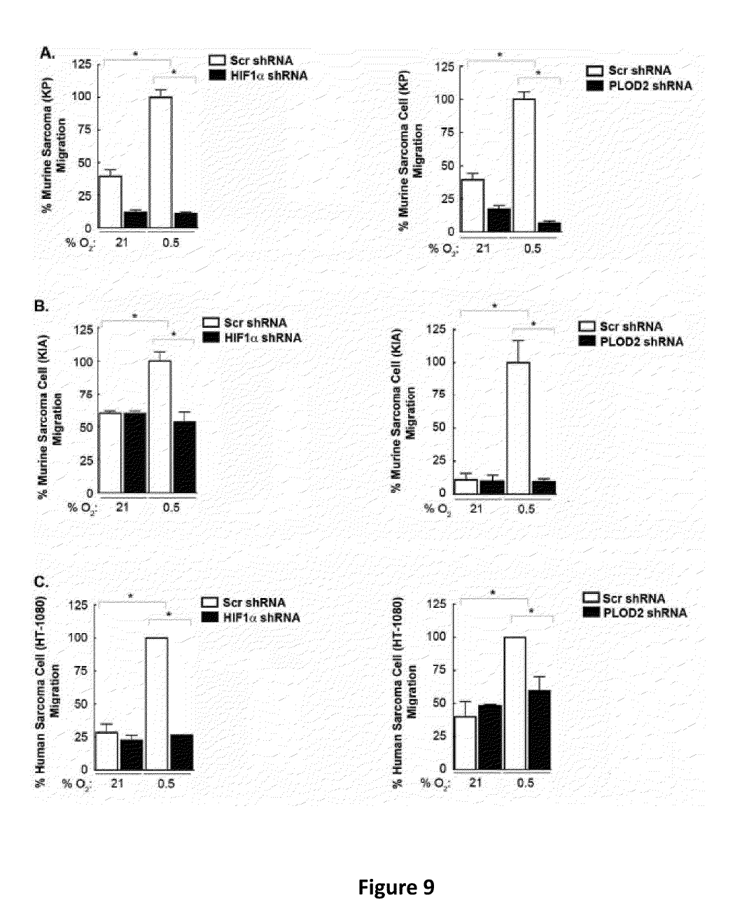

HIF1α and collagen deposition are known to promote metastasis by regulating tumor cell migration and invasion (Erler et al., 2006, Nature 440(7088):1222-6; Egeblad et al., 2010, Curr Opin Cell Biol 22(5):597-706; Erler and Giaccia, 2006, Cancer Res 66(21):10238-41; Yang and Wu, 2008, Cell Cycle 7(14):2090-6; Yang et al., 2008, Nat Cell Biol 10(3):295-305). Without wishing to be bound by any particular theory, it is believed that HIF1α regulates these processes in sarcomas through upregulation of PLOD2 transcription. Boyden chamber based migration assays, using immunoflourescent staining of migratory cell nuclei with 4′,6-diamidino-2-phenylindole (DAPI), showed that shRNA-mediated knockdown of HIF1α and PLOD2 significantly decreased sarcoma cell motility under hypoxia in KP (FIG. 9A), KIA (FIG. 9B), and HT-1080 cells (FIG. 9C). The HIF1α and migration findings are consistent with data presented by Kim et al., (Kim et al., 2013, Int. J Cancer 132(1):29-41).

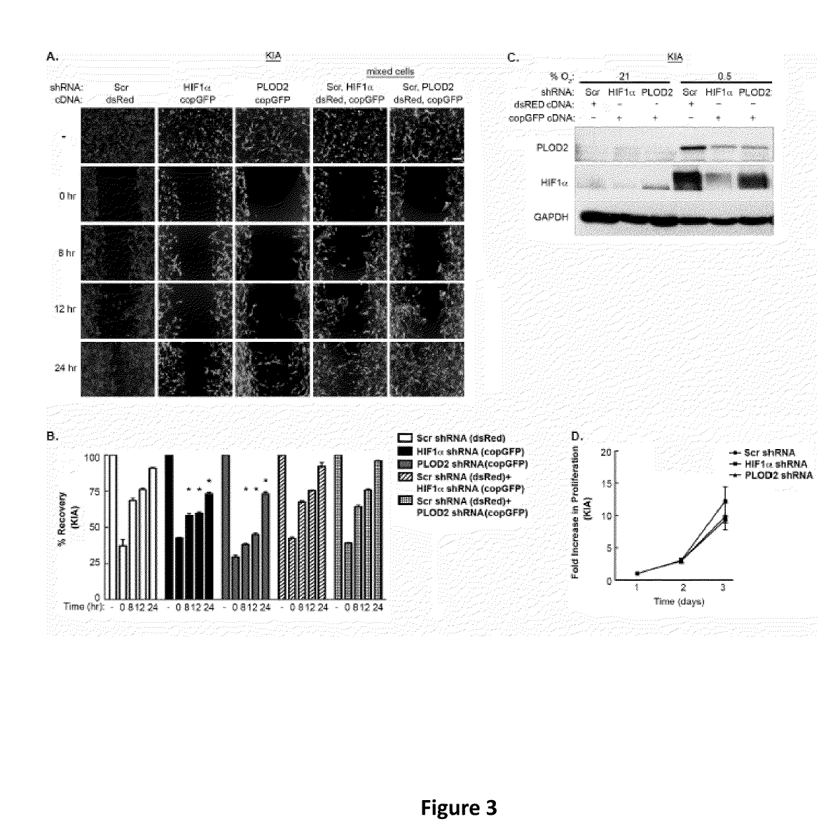

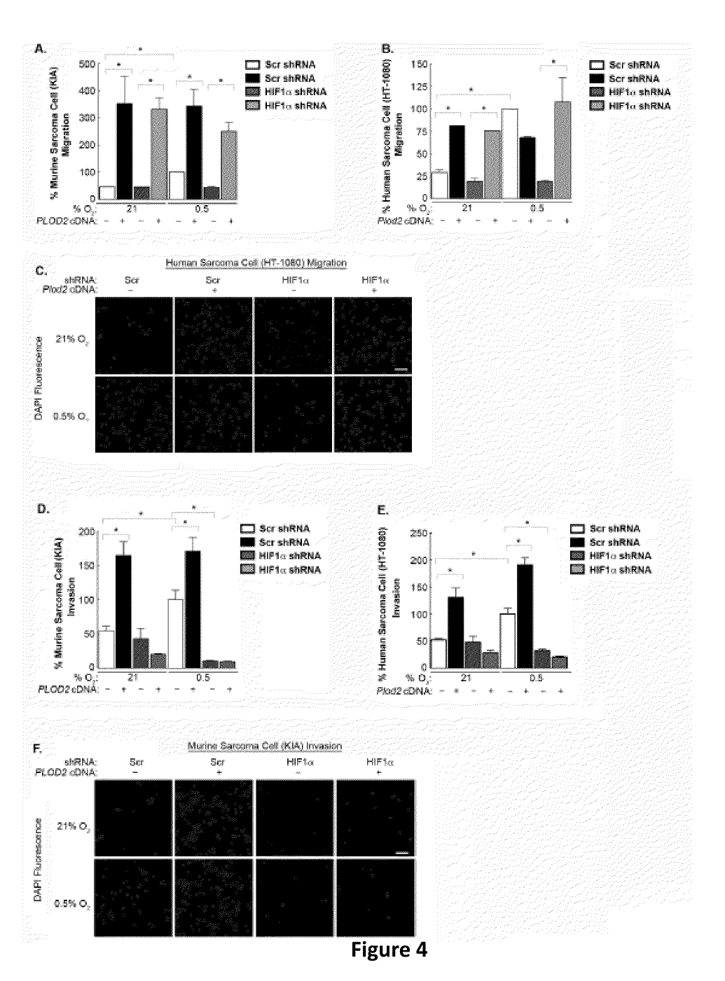

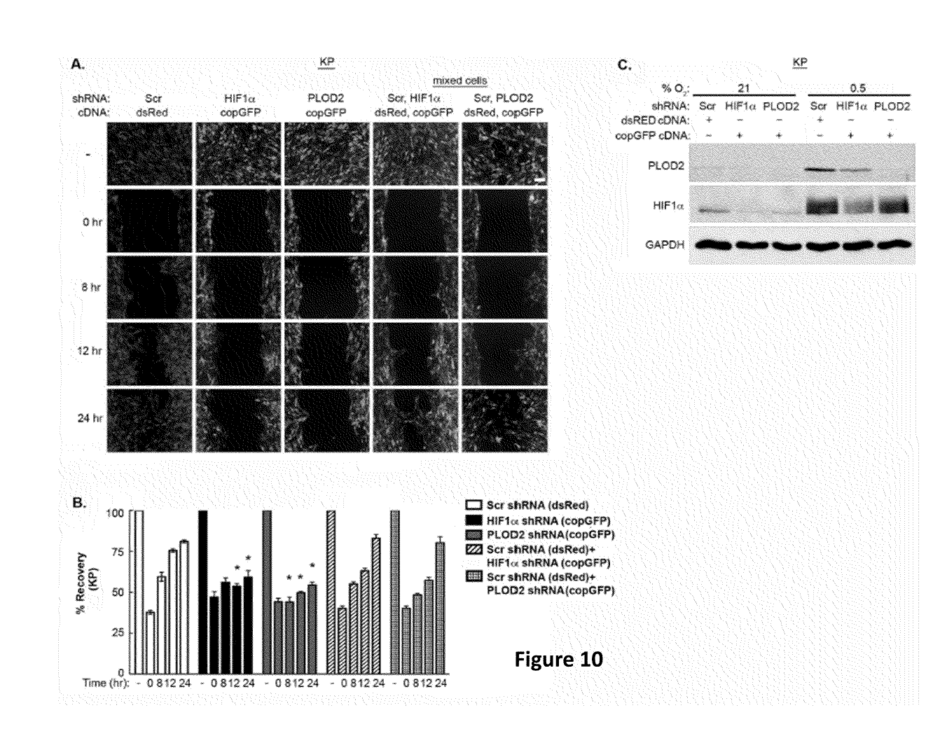

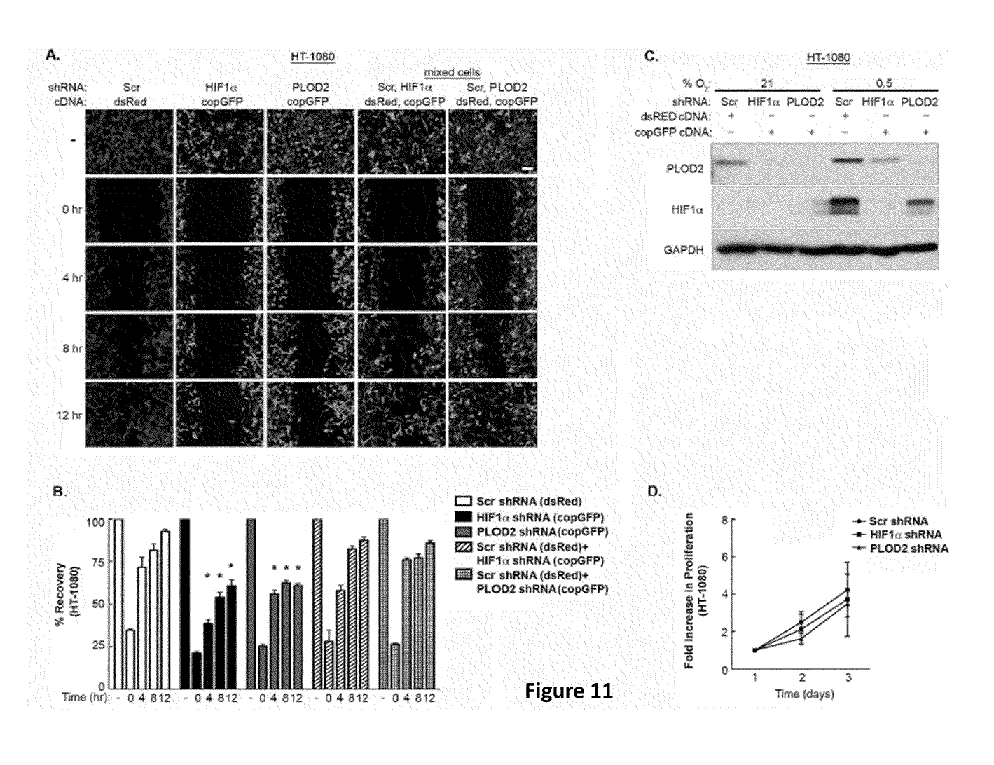

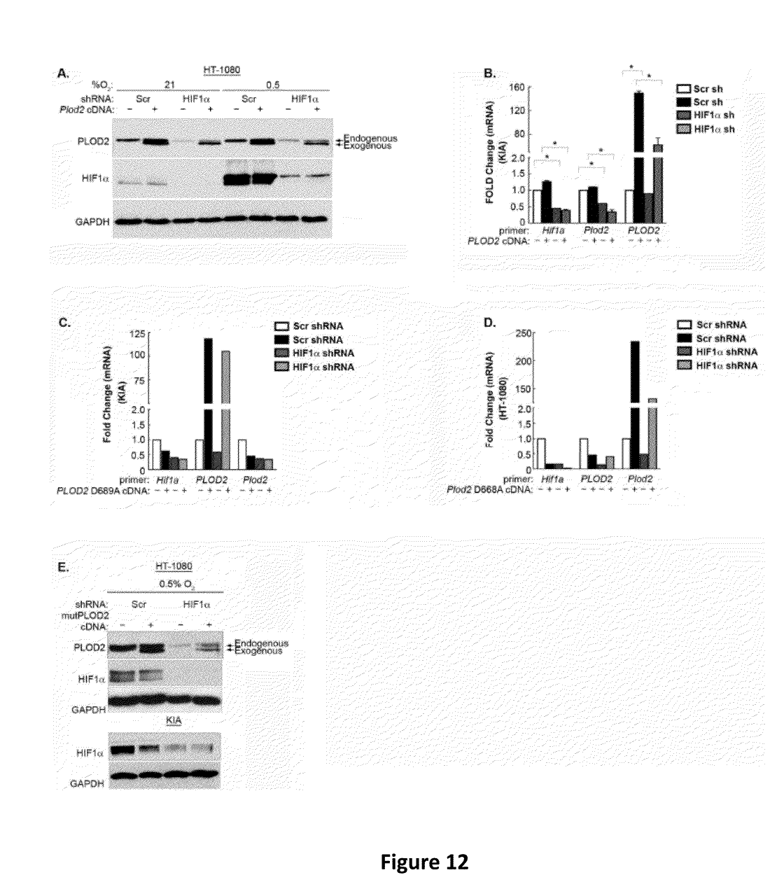

It is well established that HIF1α can influence cell migration by modulating multiple cell-intrinsic effectors, including the expression of Snail and Twist (Yang and Wu, 2008, Cell Cycle 7(14):2090-6; Yang et al., 2008, Nat Cell Biol 10(3):295-305; Mak et al., 2010, Cancer Cell 17(4):319-32). Without wishing to be bound by any particular theory, it is believed that if altering extracellular collagen deposition was the primary effect of HIF1α on sarcoma cell migration, then the presence of wild type sarcoma cells should rescue defects in matching HIF1α-deficient cells in in vitro migration assays. To test this directly, scratch migration assays was performed using stable Scr, HIF1α-deficient, and PLOD2-deficient cells transduced with lentivirus bearing a dsRed expression vector (Scr shRNA) or a copGFP expression vector (HIF1α shRNA, PLOD2 shRNA). Migration of HIF1α-deficient copGFP+ cells and PLOD2-deficient copGFP+ cells was significantly delayed compared to control Scr dsRed cells in the KIA (FIG. 3 A,B), KP (FIG. 10A,B), and HT-1080 cell lines (FIG. 11A,B). However, when control and knockdown cells were mixed together, migration of both HIF1α-deficient and PLOD2-deficient cells were restored to control levels. Expression of copGFP and dsRED did not affect the ability of HIF1α to modulate PLOD2 levels in any of the three cell types (FIG. 3C, FIG. 10C, and FIG. 11C). Importantly, deletion of HIF1α and PLOD2 did not effect proliferation in these cell lines (FIG. 3D and FIG. 11D). These data indicate that HIF1α drives sarcoma cell migration in a cell-extrinsic manner, possibly through PLOD2-mediated effects on collagen modification. To determine whether HIF1α-mediated migration and invasion were PLOD2-dependent, PLOD2 was ectopically expressed in HIF1α-deficient KIA (FIG. 4A) and HT-1080 cells (FIG. 4B,C). HIF1α-deficient HT-1080 and KIA cells were transduced with control lentivirus or lentivirus bearing a wild-type PLOD2 expression vector. Western blot analysis showed endogenous and exogenous PLOD2 levels, as well as the efficacy of HIF1α inhibition (FIG. 12A). Murine Plod2 was expressed in human HT-1080 cells, and human Plod2 in murine KIA cells, making it possible to evaluate changes in endogenous and ectopic PLOD2 mRNA levels by qRTPCR using species-specific primers (FIG. 12B). PLOD2 expression rescued cell migration in HIF1α-deficient cells under hypoxic conditions and also stimulated migration in normoxic cells (FIG. 4A,B,C). Interestingly, PLOD2 did not rescue invasion in KIA (FIG. 4D,F) or HT-180 cells (FIG. 4E) suggesting that the HIF1α/PLOD2 pathway primarily regulates sarcoma cell motility. As discussed elsewhere herein, neither HIF1α nor PLOD2 knockdown had any significant effect on proliferation in either cell type over a 3-day period (FIG. 3D and FIG. 11D). Without wishing to be bound by any particular theory, it is believed concluded that although HIF1α regulates multiple aspects of metastasis (migration, invasion), HIF1α-dependent modulation of PLOD2 levels selectively impacts sarcoma cell motility.

### PLOD2 Lysyl Hydroxylase Activity is Required for Sarcoma Cell Migration

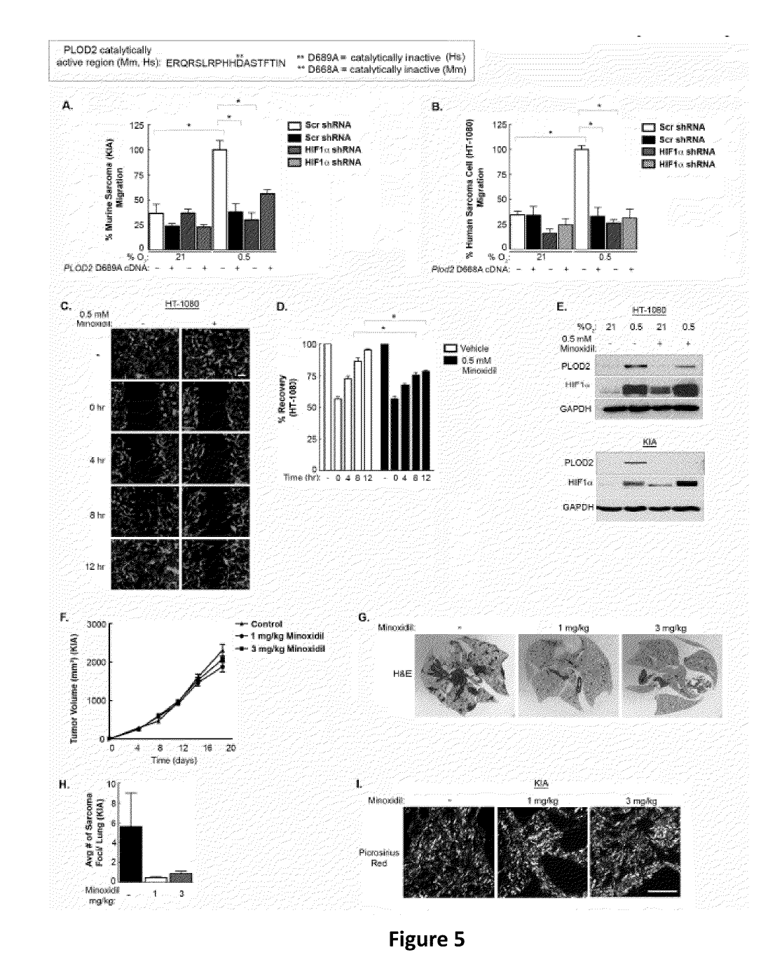

PLOD2 lysyl hydroxylase activity is dependent upon association with several essential cofactors, including Fe2+ and 2-oxoglutarate, which requires a conserved aspartate residue (D689 in human PLOD2; D668 in mouse), and mutation of these amino acids inactivates PLOD2 (Rautavuoma et al., 2002, J Biol Chem 277(25):23084-91; Pirskanen et al., 1996, J Biol Chem 271(16):9398-402). Using site-directed mutagenesis, inactive PLOD2 (D689A, D668A) was generated to determine if the enzymatic activity of PLOD2 was essential for its ability to rescue cell migration in HIF1α-deficient cells. Migration assays were performed on stable Scr control and HIF1α-deficient HT-1080 and KIA cells that had also been transduced with lentivirus bearing a mutant PLOD2 expression vector. It was observed that expression of inactive PLOD2 mutants failed to rescue migration in HIF1α-deficient KIA (FIG. 5A) and HT-1080 cells (FIG. 5B). Furthermore, mutant PLOD2 behaves as a dominant negative in KIA and HT-1080 cells, suppressing hypoxia-induced migration. Interestingly, significant overexpression of mutant PLOD2 modestly inhibited endogenous PLOD2 and HIFla levels as shown by qRT-PCR (FIG. 12C,D) and western blot analysis (FIG. 12E).

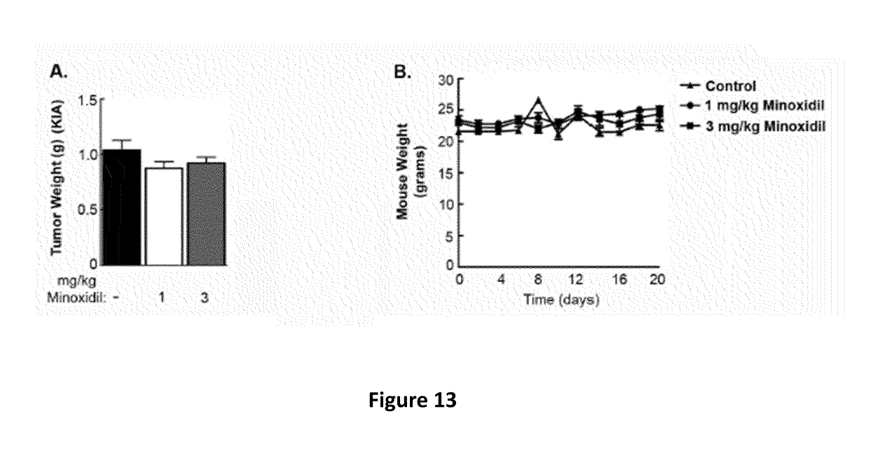

To confirm that PLOD2 is required for sarcoma cell migration, pharmacological inhibitor of PLOD2 expression, Minoxidil, was used (Zuurmond et al., 2005, Matrix Biol 24(4):261-70). Minoxidil treatment (0.5 mM) for 48 hours significantly reduced HT-1080 cell migration (FIG. 5C,D), concomitant with reduced PLOD2 protein levels as shown by western blot analysis of HT-1080 and KIA cells (FIG. 5E). Interestingly, Minoxidil increased HIFα levels, but not cell migration, supporting the conclusion that HIF1α-dependent induction of PLOD2 lysl hydroxylase activity is required for sarcoma cell migration. To determine the physiological importance of Minoxidil as a sarcoma metastasis inhibitor, allografts of subcutaneously injected KIA cells were generated in nude mice and immediately began injections of PBS, 1 mg/kg Minoxidil, or 3 mg/kg Minoxidil every other day for 3 weeks. Minoxidil had no effect on primary tumor volume, tumor weight, or overall animal health/weight (FIG. 5F and FIG. 13). However, Minoxidil treatment reduced the number of pulmonary metastases (FIG. 5G,H). Consistent with these observations in the autochthonous model, Minoxidil treated tumors contained relatively organized collagen compared with control treated tumors (FIG. 5I). These data demonstrate the potential usefulness of Minoxidil as a treatment for pre-metastatic sarcoma.

### In Vivo Metastasis Requires HIF1α/PLOD2-Mediated Collagen Production

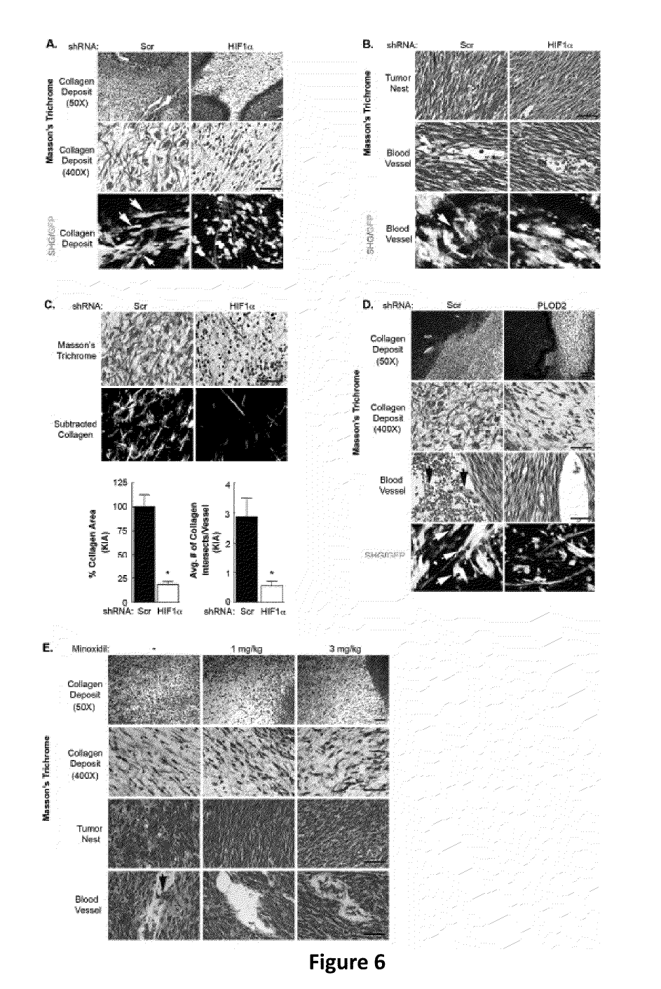

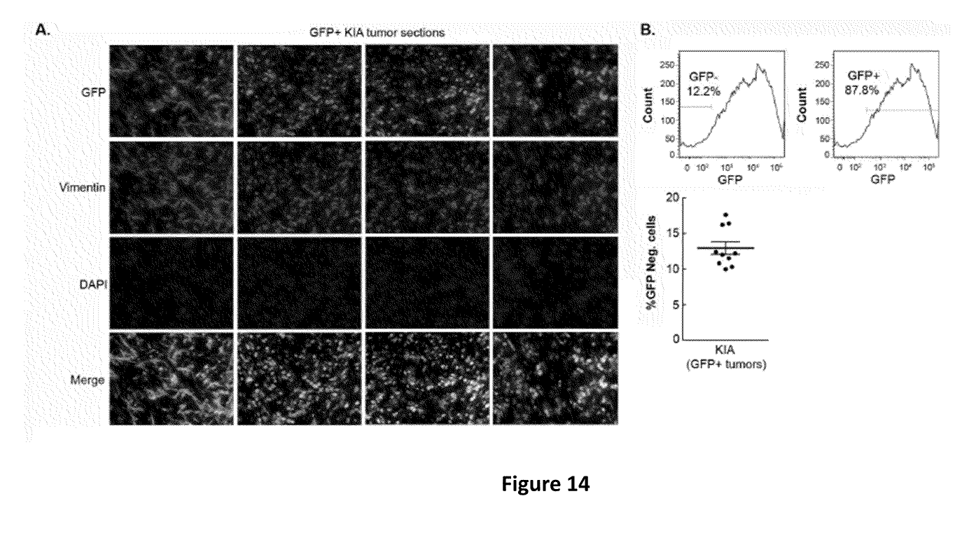

Experiments were performed to test the hypothesis that HIF1α-dependent PLOD2 expression and collagen modification are required for cell migration and metastasis in vivo using the KIA tumor transplant model of metastatic UPS. Staining of HIF1α-deficient tumor sections with Masson's Trichrome revealed a significant change in collagen staining, confirming that collagen was altered in the absence of HIF1α (FIG. 6A,B). Collagen was quantified using ImagePro7 software, which revealed a dramatic shift in collagen density in HIF1α-deficient tumors compared with control tumors (FIG. 6C). Intriguingly, HIF1α-deficient tumor cells appear smaller and rounder than control cells, which may reflect their relative inability to associate with and migrate along collagen fibers. To investigate this possibility, experiments were designed to perform second harmonic generation (SHG) analysis of explanted control and HIF1α-deficient tumors (FIG. 6A,B). SHG imaging permits the co-visualization of endogenous collagen and GFP+ tumor cells in live tissue. In control tumors, areas of highly branched/complex collagen were identified and it was observed that GFP+ tumor cells associated closely with collagen and their morphology was elongated as they adhered to the fibers. In contrast, HIF1α-deficient tumors lacked complex highly branched collagen deposits and the tumor cells did not elongate or associate with the collagen that remained (FIG. 6A-C). PLOD2-deficient tumors also possess defects in collagen deposition and cellular morphology, phenocopying what is seen when HIF1α is silenced (FIG. 6D). In many epithelial tumors, mesenchymal cells like fibroblasts are recruited and subsequently secrete collagen. However, in mesenchymal lesions the tumor cells themselves perform this function. Therefore, experiments were performed to determine whether a fibroblast population depositing additional collagen was present in the sarcoma model. Immunofluorescence analysis of GFP+ KIA tumor sections stained for GFP and the mesenchymal marker, Vimentin, showed that a small percentage of cells in the tumor were GFP−; Vimentin+ as expected for an infiltrating fibroblast population (FIG. 14A). This population was quantified by flow cytometry using single cells suspensions of dissociated GFP+tumors. Roughly 12% of cells in these tumors were GFP− infiltrating cells (FIG. 14B). These data suggest that the tumor cells themselves deposit the majority of collagen secreted in sarcomas.

An important aspect of collagen-associated tumor cell migration is the ability of the collagen network to deliver cells to the vasculature. To determine if this process is compromised in HIF1α-deficient sarcomas, the instances where collagen invades blood vessels in the tumors was quantified. In the absence of HIF1α, the loss of collagen at the blood vessels was severe (FIG. 6B). These findings are consistent with the overall conclusion that loss of HIF1α prevents tumor cells from migrating to vessels and escaping the primary lesion. PLOD2 ablation results in defects in collagen/vessel association similar to that of HIF1α-deficiency (FIG. 6B,D). Masson's Trichrome staining of Minoxidil treated tumors showed results similar to that of HIF1α and PLOD2-deficient tumors. Deposited collagen appears thinner and did not penetrate the vasculature, preventing tumor cells from using the collagen “highway” to disseminate to distant sites (FIG. 6E).

### PLOD2 Expression Rescues In Vivo Metastasis Arising HIF1α-Deficient Primary Sarcomas

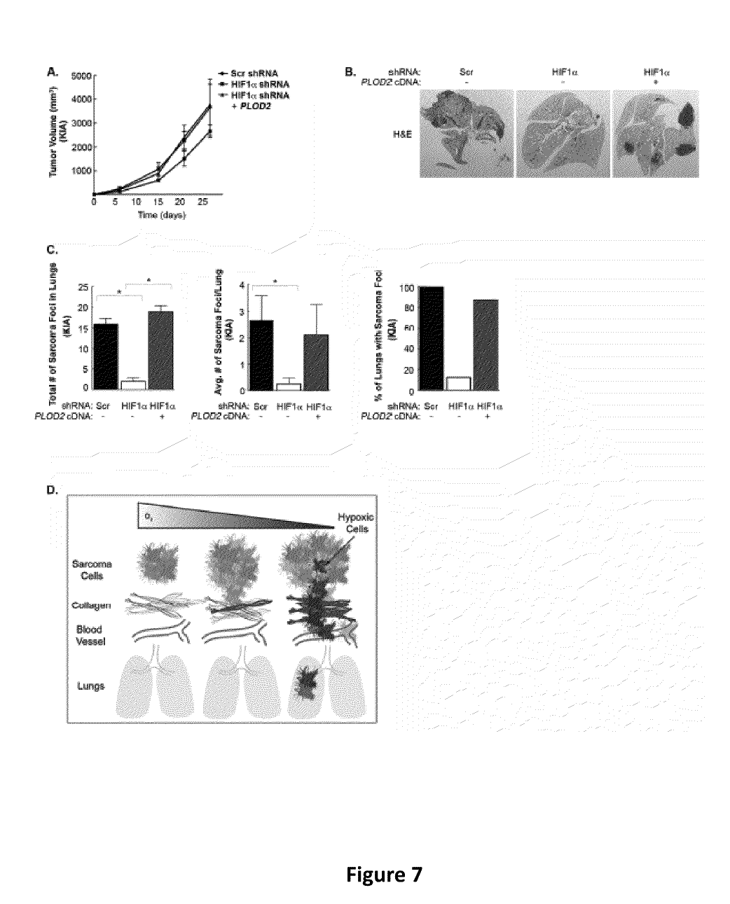

In order to clearly show that HIF1α-mediated regulation of PLOD2 is essential for metastasis, an in vivo rescue experiment was performed in which wild-type PLOD2 was ectopically expressed in HIF1α-deficient tumors. Expression of PLOD2 rescued metastatic potential in KIA tumors (FIG. 7B,C), while having no reproducible effect on primary tumor volume (FIG. 7A). Together these data show that HIF1α-mediated metastasis is dependent upon PLOD2 modification of collagen networks.

### HIF1α-Dependent PLOD2 Expression is Required for Metastasis.

Soft tissue sarcomas are a highly complex set of malignancies, comprising more than 50 histologically distinct sub-types associated with genetic alterations in diverse molecular pathways (Taylor et al., 2011, Nat Rev Cancer 11(8):541-57). Although lethal metastases, particularly to the lung, are a common occurrence in sarcoma patients, the molecular mechanisms regulating this process are largely unknown. Primary sarcomas are noted for extensive fibrosis and deposition of extracellular matrix components, a feature that has been associated with metastatic potential in numerous cancers (Noda et al., 2012, Liver Int 32(1):110-8; Akiri et al., 2003, Cancer Res 63(7):1657-66; Colpaert et al., 2001, Am J Surg Pathol 25(12):1557-8; Colpaert et al., 2001, Histopathology 39(4):416-25; Hasebe et al., 1997, Jpn J Cancer Res 88(6):590-9; Martin and Boyd, 2008, Breast Cancer Res 10(1):201). Although many details are incompletely understood, it is clear that metastatic tumor cells can associate physically with dense collagen networks in solid tumors, and migrate along this collagen “highway” toward vascular tissues (Condeelis et al., 2003, Nat Rev Cancer 3(12):921-30; Roussos et al., 2011, Nat Rev Cancer 11(8):573-87), through which they ultimately disseminate and colonize distant organs. Therefore, therapeutic manipulation of collagen modification/organization in primary sarcomas, as well as other tumors, could have significant impact on an early, proximal step in metastasis, which remains the leading cause of cancer-related death.

Low intratumoral O₂ levels and HIF1α a expression are key predictors of metastatic potential in sarcomas. Although previous microarray analyses revealed elevated HIF1α and PLOD2 expression in sarcoma patient samples (Detwiller et al, 2005, Cancer Res 65(13):5881-9), the mechanisms by which these genes regulate sarcoma progression and/or metastasis were unclear. In this study, it has been demonstrated that HIF1α and PLOD2 expression levels are preferentially elevated in primary human sarcomas that subsequently metastasized, suggesting they regulate one or more aspect of tumor cell dissemination. Using independent murine sarcoma models, it has been determined that HIF1α-dependent PLOD2 expression is required for deposition of the disorganized collagen required to facilitate tumor cell migration in primary tumors, as well as pulmonary metastasis. In vitro assays revealed that the HIF1α/PLOD2 pathway specifically regulates sarcoma cell migration in a collagen-dependent manner. Finally, ectopic expression of PLOD2 rescues both cell migration and metastatic potential in HIF1α-deficient cells and tumors. Collectively, the data presented herein indicate that HIF1α-dependent PLOD2 expression is essential for hypoxia-mediated collagen disorganization and metastasis in sarcomas (see model in FIG. 7D). Unexpectedly, the results demonstrate that disorganized/immature collagen take on a denser structure and are alternately modified in a way that supports tumor cell adherence and migration. Deletion of HIF1α/PLOD2 restores collagen modification and organization, preventing its association with tumor cells.

It is important to note that HIF1α regulates multiple, distinct pathways associated with metastasis in various tumor models. For example, HIF1α reduces E-cadherin expression and promotes invasiveness and the epithelial to mesenchymal transition (EMT) in renal cancers (Krishnamachary et al., 2006, Cancer Res 66(5):2725-31). Moreover, HIF1α a enhances metastasis by regulating TWIST1 transcription in head and neck squamous cell cancers (Yang and Wu, 2008, Cell Cycle 7(14):2090-6; Yang et al., 2008, Nat Cell Biot 10(3):295-305). HIF1α has also been shown to play important cell-extrinsic roles in breast cancer models, where it induces expression of the ECM modifying enzyme LOX, which promotes effective metastasis by modifying the pre-metastatic niche (Erler et al., 2006, Nature 440(7088):1222-6; Erler and Giaccia, 2006, Cancer Res 66(21):10238-41). Additionally, HIF1α-dependent expression of angiopoietin-like 4 and L1CAM was recently shown to promote breast cancer metastasis via control tumor cell invasion of the vasculature (Zhang et al., 2012, Oncogene 31(14):1757-70). In contrast, HIF1α-dependent PLOD2 expression in primary sarcomas has a profound effect on extracellular collagen networks, which in turn regulate tumor cell migration and the initiation of metastasis. Interestingly, PLOD2 has been recently identified as a novel prognostic factor in hepatocellular carcinoma, in which it is associated with disease recurrence and intrahepatic metastases (Noda et al., 2012, Liver Int 32(1):110-8). Although previous work from multiple groups has highlighted (Condeelis et al., 2003, Nat Rev Cancer 3(12):921-30; Roussos et al., 2011, Nat Rev Cancer 11(8):573-87) the importance of collagen as a scaffold that supports the migration of metastatic tumor cells, the cellular processes that create these collagen deposits are relatively understudied.

The results presented herein demonstrate the role of HIF1α and PLOD2 in sarcoma. It was shown that HIF1α-dependent upregulation of PLOD2, but not LOX, was observed in metastatic human sarcomas, and was essential for the creation of collagen networks in primary murine tumors and subsequent metastasis to the lung. Importantly, Minoxidil-mediated PLOD inhibition decreased pulmonary metastasis in the murine allograft sarcoma model, suggesting that PLOD inhibition may prove a useful therapeutic intervention. The results presented herein indicate that intratumoral hypoxia and HIF1α-dependent PLOD2 transcription promote sarcoma metastasis by modifying the collagen component of the ECM in primary tumors, and stimulating sarcoma cell migration. Furthermore, these data indicate that HIF1α confers distinct, tumor type-dependent effects on metastasis. Specifically, whereas HIF1α-driven LOX expression has been shown to modify the premetastatic niche in breast cancers, and possibly other carcinomas, PLOD2 modifies the collagen network in primary sarcomas, with consequent effects on tumor cell migration and metastasis. Also, the results presented herein demonstrate that PLOD2 is a credible and druggable therapeutic target in pre-metastatic sarcoma.

As hypoxia and HIF expression are important prognostic indicators in many solid tumors, conclusions drawn from the current studies may be applicable in multiple tumor contexts, although other collagen modifying enzymes, such as PLOD1 or LOX, may also contribute in other tumor types. The present experiments used the PLOD2 inhibitor, Minoxidil, to address the importance of PLOD2 in tumor cell migration and in vivo pulmonary metastases.

Although metastases are responsible for the vast majority of cancer-associated deaths, there are very few therapeutic approaches that specifically target metastasis in any tumor model. The present invention provides compositions and methods for compromising the ECM network as a type of anti-tumor therapy. The compositions and methods of the invention can be used in conjunction with the current standard of therapy.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for interfering with at least one of HIF1α and a collagen modifying enzyme, the method comprising administering to a subject in need thereof an effective amount of a composition comprising an inhibitor of at least one of HIF1α and a collagen modifying enzyme.

2. The method of claim 1, wherein the collagen modifying enzyme is selected from the group consisting of procollagen-lysine 5-dioxygenase 1 (PLOD1); procollagen-lysine, 2-oxoglutarate 5-dioxygenase 2 (PLOD2); procollagen-lysine 5-dioxygenase 3 (PLOD3) and any combination thereof.

3. The method of claim 1, wherein interfering with a collagen modifying enzyme comprises one or more of the level of the collagen modifying enzyme and the activity of the collagen modifying enzyme.

4. The method of claim 1, wherein the inhibitor prevents the transcription of the collagen modifying enzyme gene or translation of the collagen modifying enzyme mRNA.

5. The method of claim 1, wherein the inhibitor interferes with the activity of the collagen modifying enzyme.

6. The method of claim 1, wherein the inhibitor is selected from the group consisting of a small interfering RNA (siRNA), a microRNA, an antisense nucleic acid, a ribozyme, an expression vector encoding a transdominant negative mutant, an antibody, a peptide and a small molecule.

7. The method of claim 1, wherein the inhibitor is minoxidil or a salt or chemical analog thereof.

8. The method of claim 1, wherein the collagen modifying enzyme is associated with at least one of cancer metastasis, cancer cell growth, cancer invasion, and cancer angiogenesis.

9. The method of claim 1, wherein the collagen modifying enzyme is associated with one or more of scarcoma metastasis, lung metastasis, and pulmonary metastasis.

10. A system for diagnosing the progression of cancer in a subject, comprising a probe capable of detecting the expression of one or more of HIF1α and a collagen modifying enzyme in a subject.

11. The system of claim 10, wherein detecting the expression of the collagen modifying enzyme a subject comprises detecting expression of the collagen modifying enzyme mRNA in a subject.

12. The system of claim 10, wherein detecting the expression of the collagen modifying enzyme mRNA in a subject comprises detecting expression of collagen modifying enzyme the mRNA in a tumor cell or a mesenchymal cell.

13. The system of claim 10, wherein detecting the expression of the collagen modifying enzyme in a subject comprises detecting expression of the collagen modifying enzyme protein in a subject.

14. The system of claim 10, wherein detecting the expression of the collagen modifying enzyme protein in a subject comprises detecting expression of the collagen modifying enzyme protein in a tumor cell or a mesenchymal cell.

15. The system of claim 10, wherein the probe comprises a nucleic acid or a protein.

16. The system of claim 15, further comprising a detector capable of detecting the interaction of the probe with a target associated with the collagen modifying enzyme expression.

17. The system of claim 16, wherein the collagen modifying enzyme is expressed in non-metastatic cells at a first amount and the collagen modifying enzyme is expressed in metastatic cells at a second amount that is greater than the first amount.

18. A method for diagnosing the progression of cancer in a subject, the method comprising detecting one or more of expression of a collagen modifying enzyme in a subject and activity of a collagen modifying enzyme in a subject; and diagnosing the progression of cancer in a subject.

19. The method of claim 18, wherein the collagen modifying enzyme is selected from the group consisting of procollagen-lysine 5-dioxygenase 1 (PLOD1); procollagen-lysine 2-oxoglutarate 5-dioxygenase 2 (PLOD2); procollagen-lysine 5-dioxygenase 3 (PLOD3) and any combination thereof.

20. A method for treating a neoplastic disease comprising administering to a subject an effective amount of a composition comprising an inhibitor of one or more of HIF1α and a collagen modifying enzyme.

21. The method of claim 20, further comprising at least one of reducing the metastasis of a cancer in a subject, reducing the cell growth of a cancer in a subject, reducing the invasiveness of a cancer in a subject, or reducing the angiogenesis of a cancer in a subject.

22. The method of claim 20, wherein the neoplastic disease is a sarcoma.

23. The method of claim 20, wherein the inhibitor is minoxidil or a salt or chemical analog thereof.

24. The method of claim 20, further comprising administering a therapeutic agent to the subject.

25. The method of claim 20, wherein the subject is a human.

26. A method for preventing a neoplastic disease from metastasizing, the method comprising administering to a subject an effective amount of a composition comprising an inhibitor of a collagen modifying enzyme.

27. The method of claim 26, wherein the neoplastic disease is a sarcoma.

28. The method of claim 26, wherein the inhibitor is minoxidil or a salt or chemical analog thereof.

29. The method of claim 26, further comprising administering a therapeutic agent to the subject.

30. The method of claim 26, wherein the subject is a human.
